(19) United States
(12) Patent Application Publication
(10) Pub. No.: US 2019/0093172 A1
(43) Pub. Date: Mar. 28, 2019

(54) PLASMA-BASED DETECTION OF ANAPLASTIC LYMPHOMA KINASE (ALK) NUCLEIC ACIDS AND ALK FUSION TRANSCRIPTS AND USES THEREOF IN DIAGNOSIS AND TREATMENT OF CANCER

(71) Applicant: Exosome Diagnostics, Inc., Waltham, MA (US)

(72) Inventors: Johan Karl Olov SKOG, Charlestown, MA (US); Mikkel NOERHOLM, Martinsried (DE); James HURLEY, Waltham, MA (US); Elena CASTELLANOS-RIZALDOS, Waltham, MA (US); Kay BRINKMAN, Waltham, MA (US)

(73) Assignee: Exosome Diagnostics, Inc., Waltham, MA (US)

(21) Appl. No.: 16/092,358

(22) PCT Filed: Apr. 17, 2017
(86) PCT No.: PCT/US2017/027944

§ 371 Date: Oct. 9, 2018

Related U.S. Application Data
(60) Provisional application No. 62/322,982, filed on Apr. 15, 2016.

(51) Int. Cl. C12Q 1/6886; C12N 15/10; C12Q 1/686; G16H 50/20

(52) U.S. Cl. CPC C12Q 1/6886; C12N 15/1003; C12Q 1/686; C12Q 2600/106; C12Q 2600/158; C12Q 2600/156; C12Q 2600/118; G16H 50/20

(57) ABSTRACT

The present invention relates generally to the field of biomarker analysis, particularly determining gene expression signatures from biological samples, including plasma samples.

### RELATED APPLICATIONS

This application claims the benefit of U.S. Provisional Application No. 62/322,982, filed Apr. 15, 2016, the contents of which are incorporated herein by reference in their entirety.

### FIELD OF THE INVENTION

The present invention relates generally to the field of biomarker analysis, particularly determining gene expression signatures from biological samples, including plasma samples.

### BACKGROUND

Increasing knowledge of the genetic and epigenetic changes occurring in cancer cells provides an opportunity to detect, characterize, and monitor tumors by analyzing tumor-related nucleic acid sequences and profiles. These changes can be observed by detecting any of a variety of cancer-related biomarkers. Various molecular diagnostic assays are used to detect these biomarkers and produce valuable information for patients, doctors, clinicians and researchers. So far, these assays primarily have been performed on cancer cells derived from surgically removed tumor tissue or from tissue obtained by biopsy.

However, the ability to perform these tests using a bodily fluid sample is oftentimes more desirable than using a patient tissue sample. A less invasive approach using a bodily fluid sample has wide ranging implications in terms of patient welfare, the ability to conduct longitudinal disease monitoring, and the ability to obtain expression profiles even when tissue cells are not easily accessible.

Accordingly, there exists a need for new, noninvasive methods of reliably detecting biomarkers, for example, biomarkers in plasma microvesicles, to aid in diagnosis, prognosis, monitoring, or therapy selection for a disease or other medical condition.

### SUMMARY OF THE INVENTION

The present invention is in the technical field of biotechnology. More particularly, the present invention is in the technical field of molecular biology.

In molecular biology, molecules, such as nucleic acids, can be isolated from human sample material, such as plasma and other biofluids, and further analyzed with a wide range of methodologies.

Human biofluids contain cells and also cell free sources of molecules shed by all cells of the body. Cell free sources include extracellular vesicles (EVs) and the molecules carried within (e.g. RNA, DNA, lipids, small metabolites and proteins) and also cell free DNA, which is likely to be derived from apoptotic and necrotic tissue.

Since cell free nucleic acids, such as the RNA contained in exosomes and other EVs (exoRNA), DNA contained in exosomes and other EVs (exoDNA), free circulating or cell free DNA (cfDNA) are shed not only by normal somatic cells, but also aberrant cancer cells, an isolation of exosomal nucleic acids and DNA from human blood samples can reveal the existence and type of cancer cells in a patient.

Non-small cell lung cancer (NSCLC) comprises ˜85% of all diagnosed lung cancers. Obtaining tissue biopsies from NSCLC is challenging, and as many as 30% of patients have no tissue for molecular analysis of genes, therefore monitoring the mutations in blood as a liquid biopsy have proven useful. The compositions and methods provided herein use the information derived from cellular living processes such as exosomal RNA (exoRNA) release, which leads to an extremely sensitive assay. It is understood that while the examples provided herein demonstrate the isolation of exoRNA, the methods and kits provided herein are useful for co-isolating any combination of exosomal nucleic acids, e.g., exoRNA and/or exoDNA, found in the sample.

The existence and quantity of an ALK fusion transcript, e.g., an EML-ALK fusion transcript, in a patient can be used to guide or select the treatment options.

Here we describe the application of a PCR-based assay on exoRNA and isolated from human biofluids that detects an ALK fusion transcript, e.g., an EML-ALK fusion transcript, with high sensitivity and specificity.

The present invention is a complete workflow from sample extraction to nucleic acid analysis using exosomal RNA. State-of-the-art machine learning and data-mining techniques are applied to the qPCR data generated by the real time instrument to discriminate between positive and negative samples or to quantify the strength of positive or negative samples.

The present disclosure provides methods of detecting one or more biomarkers in a biological sample to aid in diagnosis, prognosis, monitoring, or therapy selection for a disease such as, for example, cancer. The methods and kits provided herein are useful in detecting one or more biomarkers from plasma samples. The methods and kits provided herein are useful in detecting one or more biomarkers from the microvesicle fraction of plasma samples.

The methods and kits provided herein are useful for detecting an anaplastic lymphoma kinase (ALK) fusion transcript in a biological sample. In some embodiments, the ALK fusion transcript is an EML-ALK fusion transcript. In some embodiments, the ALK fusion transcript is an EML4-ALK fusion transcript. In some embodiments, the EML4-ALK fusion transcript is EML4-ALK v1, EML4-ALK v2, EML4-ALK v3, and any combination thereof.

The present disclosure provides methods and kits for detecting a EML4-ALK fusion transcript in a biological sample. In some embodiments, the biological sample is plasma.

The present disclosure provides a reaction designed to capture and concentrate EVs, isolate the corresponding nucleic acids, and to simultaneously detect the presence of an ALK fusion transcript, e.g., an EML-ALK fusion transcript.

Generally, the methods and kits of the disclosure include the following steps:

1) Isolation of exoRNA from a biofluid sample:
a. Binding of microvesicles and other extracellular vesicles (EVs) to columns or beads;
   i. In some embodiments, the binding step is performed using the methods as described in PCT applications WO 2016/007755 and WO 2014/107571.
b. Release from matrix using lysing conditions;
c. Isolation of total nucleic acids from lysate using silica columns or beads
   i. In some embodiments, the isolating step is performed using the methods as described in PCT applications WO 2016/007755 and WO 2014/107571;

2) Detection and quantification of one or more EML-ALK fusion transcript(s);

3) Analyzing the detected and quantified EML-ALK fusion transcript(s) using the following procedure:
a. Step 1: Each sample is checked for passing the acceptance criteria for the Sample Integrity Control and the Sample Inhibition Control.
   i. In some embodiments, the Sample Integrity Control is the expression level of the housekeeping gene RPL4 tested by qPCR.
   ii. For RPL4 the acceptance criteria are defined by a cycle threshold (CT) value ≤28.
   iii. In some embodiments, the Sample Inhibition Control is the expression level of Qbeta RNA spiked into the reverse transcription reaction of each sample and tested by qPCR.
   iv. For Qbeta RNA, the acceptance criteria are defined by a CT value ≤34 for 12,500 copies spiked into reverse transcription reaction.
b. Step 2: Each run of samples is checked for a set of Positive Amplification Controls being tested in parallel.
   i. In some embodiments, the Positive Amplification Controls are defined by 3 reference DNAs coding for EML4-ALK v1, v2 v3, 1 reference DNA coding for RPL4, 1 reference RNA coding Qbeta. These reference nucleic acids are quantified by qPCR methods.
   ii. For EML4-ALK DNA, the acceptance criteria are defined by a CT range of 22-25 for 50 copies of each DNA spiked into reverse transcription reaction.
   iii. For RPL4 DNA the acceptance criteria are defined by a CT range of 26-28 for 125,000 copies of DNA spiked into reverse transcription reaction.
   iv. For Qbeta RNA, the acceptance criteria are defined by a CT range of 28-31 for 12,500 copies of RNA spiked into reverse transcription reaction.
c. Step 3: Each run of samples is checked for a set of Negative Amplification Controls being tested in parallel.
   i. In some embodiments, the Negative Amplification Controls are defined by the same set of qPCR as for Positive Amplification Control, but water is used instead of the nucleic acid template.
   ii. As acceptance criteria, no CT value must be detected.
   iii. If all sample-internal and external controls are passed, the sample is checked for EML4-ALK 4 Step 4.
   iv. If a sample-internal or external controls fails, the sample must be reported as “Inconclusive”. If residual sample material is available, the test is repeated from Step 1.
d. Step 4: Each sample is checked for passing the acceptance criteria for expression of EML4-ALK fusion variants.
   i. For qPCR of EML4-ALK variant 1 the acceptance criteria are CT≤31
   ii. For qPCR of EML4-ALK variant 2 the acceptance criteria are CT≤32
   iii. For qPCR of EML4-ALK variant 3 the acceptance criteria are CT≤32
   iv. If a sample passes the acceptance criteria it is reported as “Positive” for this EML4-ALK variant. The presence of variants is expected to be mutually exclusive.
   v. If a sample fails the acceptance criteria for EML4-ALK it is reported as “Negative”.

In some embodiments, the isolation of exoRNA from a bodily fluid sample can include one or more optional steps such as, for example, reverse transcription of complete isolated total exoRNA, including first strand synthesis using a single or a blend of RT enzymes and oligonucleotides; use of a control of inhibition, exogenous RNA spike; and/or pre-amplification of the complete isolated and reverse transcribed material

In some embodiments, the methods provided herein employ further manipulation and analysis of the detection and quantification of an ALK fusion transcript, e.g., an EML-ALK fusion transcript. In some embodiments, the methods further include the step of using machine-learning model and statistical analysis to further analyze the detected nucleic acids.

In some embodiments, the methods and kits described herein isolate the microvesicle fraction by capturing the microvesicles to a surface and subsequently lysing the microvesicles to release the nucleic acids, particularly RNA, contained therein.

Previous procedures used to isolate and extract nucleic acids from the microvesicle fraction of a biological sample relied on the use of ultracentrifugation, e.g., spinning at less than 10,000 xg for 1-3 hrs, followed by removal of the supernatant, washing the pellet, lysing the pellet and purifying the nucleic acids, e.g., RNA on a column. These previous methods demonstrated several disadvantages such as being slow, tedious, subject to variability between batches, and not suited for scalability. The isolation and extract methods used herein overcome these disadvantages and provide a spin-based column for isolation and extraction that is fast, robust and easily scalable to large volumes.

The methods and kits isolate and extract nucleic acids, e.g., exosomal RNA from a biological sample using the following the extraction procedures described in PCT Publication Nos. WO 2016/007755 and WO 2014/107571, the contents of each of which are described herein in their entirety. Briefly, the microvesicle fraction is bound to a membrane filter, and the filter is washed. Then, a reagent is used to perform on-membrane lysis and release of the nucleic acids, e.g., exoRNA. Extraction is then performed, followed by conditioning. The nucleic acids, e.g., exoRNA, is then bound to a silica column, washed and then eluted.

In some embodiments, the biological sample is a bodily fluid. The bodily fluids can be fluids isolated from anywhere in the body of the subject, for example, a peripheral location, including but not limited to, for example, blood, plasma, serum, urine, sputum, spinal fluid, cerebrospinal fluid, pleural fluid, nipple aspirates, lymph fluid, fluid of the respiratory, intestinal, and genitourinary tracts, tear fluid, saliva, breast milk, fluid from the lymphatic system, semen, cerebrospinal fluid, intra-organ system fluid, ascitic fluid, tumor cyst fluid, amniotic fluid and combinations thereof. For example, the bodily fluid is urine, blood, serum, or cerebrospinal fluid.

The methods and kits of the disclosure are suitable for use with samples derived from a human subject. The methods and kits of the disclosure are suitable for use with samples derived from a non-human subject such as, for example, a rodent, a non-human primate, a companion animal (e.g., cat, dog, horse), and/or a farm animal (e.g., chicken).

The methods described herein provide for the extraction of nucleic acids from microvesicles. In some embodiments, the extracted nucleic acids are RNA. The extracted RNA may comprise messenger RNAs, transfer RNAs, ribosomal RNAs, small RNAs (non-protein-coding RNAs, non-messenger RNAs), microRNAs, piRNAs, exRNAs, snRNAs and snoRNAs or any combination thereof.

In any of the foregoing methods, the nucleic acids are isolated from or otherwise derived from a microvesicle fraction.

In any of the foregoing methods, the nucleic acids are cell-free nucleic acids, also referred to herein as circulating nucleic acids. In some embodiments, the cell-free nucleic acids are DNA or RNA.

In some embodiments, one or more control particles or one or more nucleic acid(s) may be added to the sample prior to microvesicle isolation and/or nucleic acid extraction to serve as an internal control to evaluate the efficiency or quality of microvesicle purification and/or nucleic acid extraction. The methods described herein provide for the efficient isolation and the control nucleic acid(s) along with the microvesicle fraction. These control nucleic acid(s) include one or more nucleic acids from Q-beta bacteriophage, one or more nucleic acids from a virus particles, or any other control nucleic acids (e.g., at least one control target gene) that may be naturally occurring or engineered by recombinant DNA techniques. In some embodiments, the quantity of control nucleic acid(s) is known before the addition to the sample. The control target gene can be quantified using real-time PCR analysis. Quantification of a control target gene can be used to determine the efficiency or quality of the microvesicle purification or nucleic acid extraction processes.

In some embodiments, the control nucleic acid is a nucleic acid from a Q-beta bacteriophage, referred to herein as “Q-beta control nucleic acid.” The Q-beta control nucleic acid used in the methods described herein may be a naturally-occurring virus control nucleic acid or may be a recombinant or engineered control nucleic acid. Q-beta is a member of the leviviridae family, characterized by a linear, single-stranded RNA genome that consists of 3 genes encoding four viral proteins: a coat protein, a maturation protein, a lysis protein, and RNA replicase. When the Q-beta particle itself is used as a control, due to its similar size to average microvesicles, Q-beta can be easily purified from a biological sample using the same purification methods used to isolate microvesicles, as described herein. In addition, the low complexity of the Q-beta viral single-stranded gene structure is advantageous for its use as a control in amplification-based nucleic acid assays. The Q-beta particle contains a control target gene or control target sequence to be detected or measured for the quantification of the amount of Q-beta particle in a sample. For example, the control target gene is the Q-beta coat protein gene. When the Q-beta particle itself is used as a control, after addition of the Q-beta particles to the biological sample, the nucleic acids from the Q-beta particle are extracted along with the nucleic acids from the biological sample using the extraction methods described herein. When a nucleic acid from Q-beta, for example, RNA from Q-beta, is used as a control, the Q-beta nucleic acid is extracted along with the nucleic acids from the biological sample using the extraction methods described herein. Detection of the Q-beta control target gene can be determined by RT-PCR analysis, for example, simultaneously with the biomarker(s) of interest (e.g., an ALK fusion transcript, e.g., an EML-ALK fusion transcript, alone or in combination with one or more additional biomarkers or other ALK fusion transcript(s), e.g., other EML-ALK fusion transcript(s)). A standard curve of at least 2, 3, or 4 known concentrations in 10-fold dilution of a control target gene can be used to determine copy number. The copy number detected and the quantity of Q-beta particle added or the copy number detected and the quantity of Q-beta nucleic acid, for example, Q-beta RNA, added can be compared to determine the quality of the isolation and/or extraction process.

In some embodiments, 50, 100, 150, 200, 250, 300, 350, 400, 450, 500, 1,000 or 5,000 copies of Q-beta particles or Q-beta nucleic acid, for example, Q-beta RNA, added to a bodily fluid sample. In some embodiments, 100 copies of Q-beta particles or Q-beta nucleic acid, for example, Q-beta RNA, are added to a bodily fluid sample. When the Q-beta particle itself is used as control, the copy number of Q-beta particles can be calculated based on the ability of the Q-beta bacteriophage to infect target cells. Thus, the copy number of Q-beta particles is correlated to the colony forming units of the Q-beta bacteriophage.

In some embodiments, the methods and kits described herein include one or more in-process controls. In some embodiments, the in-process control is detection and analysis of a reference gene that indicates plasma quality (i.e., an indicator of the quality of the plasma sample). In some embodiments, the reference gene(s) is/are a plasma-inherent transcript. In some embodiments, the reference gene(s) is/are selected from the group consisting of EML4, RPL4, NDUFA1, beta-actin, exon 7 of EGFR, ACADVL; PSEN1; ADSL; AGA; AGL; ALAD; ABCD1; ARSB; BCKDHB; BTD; CDK4; ERCC8; CLN3; CPDX; CST3; CSTB; DDB2; DLD; TOR1A; TAZ; EMD; ERCC3; ERCC5; ERCC6; ETFA; F8; FECH; FH; FXN; FUCA1; GAA; GALC; GALT; GBA; GBEl; GCDH; GPI; NR3C1; GSS; MSH6; GUSB; HADHA; HMBS; HMGCL; HPRT1; HPS1; SGSH; INSR; MEN1; MLH1; MSH2; MTM1; MTR; MUT; NAGLU; NF1; NF2; NPC1; OAT; OCRL; PCCA; PDHA1; PEPD; PEX12; PEX6; PEX7; PGK1; PHKA2; PHKB; PKD1; PLOD1; PMM2; CTSA; PPDX; PTEN; PTS; PEX2; PEX5; RB1; RPGR; ATXN1; ATXN7; STS; TCOF1; TPI1; TSC1; UROD; UROS; XPA; ALDH3A2; BLMH; CHM; TPP1; CYB5R3; ERCC2; EXT2; GM2A; HLCS; HSD17B1; HSD17B4; IFNGR1; KRT10; PAFAH1B1; NEU1; PAFAH2; PSEN2; RFX5; SOD1; STK11; SUOX; UBE3A; PEX1; APP; APRT; ARSA; ATRX; GALNS; GNAS; HEXA; HEXB; PCCB; PMS1; SMPD1; TAP2; TSC2; VHL; WRN; GPX1; SLC11A2; IFNAR1; GSR; ADH5; AHCY; ALDH2; ALDH9A1; BCKDHA; BLVRB; COMT; CRAT; CYP51A1; GART; GGCX; GRINA; GSTM4; GUK1; IGF2R; IMPDH2; NR3C2; NQO2; P4HA1; P4HB; PDHB; POLR2A; POLR2B; PRIM2; RPL4; RPL5; RPL6; RPL7A; RPL8; RPL11; RPL23; RPL19; RPL22; RPL23A; RPL17; RPL24; RPL26; RPL27; RPL30; RPL27A; RPL31; RPL32; RPL34; RPL35A; RPL37A; RPL36AL; ITSN1; PRKCSH; REEP3; NKIRAS2; TSR3; ZNF429; SMAD5; STX16; C16orf87; LSS; UBE2W; ATP2C1; HDGFRP2; UGP2; GRB10; GALK2; GGA1; TIMM50; MED8; ALKBH2; LYRM5; ZNF782; MAP3K15; MED11; C4orf3; RFWD2; TOMM5; C8orf82; PIM3; TTC3; PPARA; ATP5A1; ATP5C1; PLEKHAl; ATP5D; ATE1; USP16; EXOSC10; GMPR2; NT5C3; HCFC1R1; PUS1; ATP5G1; ECHDC1; ATP5G2; AFTPH; ANAPC11; ARL6IP4; LCLAT1; ATP5G3; CAPRIN2; ZFYVE27; MARCH8; EXOSC3; GOLGA7; NFU1; DNAJB12; SMC4; ZNF787; ZNF280D; BTBD7; TH005; CBY1; PTRH1; TWISTNB; SMAD2; C11orf49; HMGXB4; UQCR10; SMAD1; MAD2L1BP; ZMAT5; BRPF1; ATP5J; RREB1; MTFP1; OSBPL8; ATP5J2; RECQL5; GLE1; ATP5H; STRADA; ERLIN2; NHP2L1; BICD2; ATP5S; HNRNPD; MED15; MANBAL; PARP3; OGDH; CAPNS1; NOMO2; ALG11; QSOX1; ZNF740; RNASEK; SREBF1; MAGED1; HNRNPL; DNM2; KDM2B; ZNF32; MTIF2; LRSAM1; YPEL2; NEURL4; SF3A1; MARCH2; PKP4; SF3B1; VPS54; NUMB; SUMO1; RYK; IP6K2; JMJD8; C3orf37; IP6K1; ERBB2IP; LRRC37A2; SIAH1; TSPAN17; MAPKAP1; WDR33; ARHGAP17; GTDC1; SLC25A25; WDR35; RPS6KA4; UHRF1BP1L; RPS4X; GOSR1; ALG8; SDCBP; KLHL5; ZNF182; ZNF37A; SCP2; ZNF484; L3MBTL3; DEPDC5; CACYBP; SPOP; METTL13; IFRD1; GEMIN7; EI24; RWDD1; TULP4; SMARCB1; LMBRD2; CSDE1; SS18; IRGQ; TFG; BUB3; CEPT1; COA5; CNOT4; TTC32; C18orf25; CISD2; CGGBP1; LAMTOR4; BCAP29; SLC41A3; SEPT2; TMEM64; MXI1; USP20; NUPL1; TPST2; PICALM; CCBL2; THAP7; TFIP11; C6orf1; PPP1CA; WDR89; ZNF121; FNIP1; C6orf226; CCT3; NIPA2; CUL4A; TCP1; STK16; RCHY1; CKAP5; RPS5; GEMIN2; CCT6A; PPP2CB; CCT7; VWA8; BRD9; KIAA0930; ZCCHC11; C12orf29; KIAA2018; VPS8; TMEM230; ANKRD16; SSBP3; ZNF655; C20orf194; FAM168B; DALRD3; SSBP4; KDM1A; RPS6; ZNF766; TTC7B; RNF187; IBA57; ERCC6L2; RAP1A; TNK2; RAP1B; GLT8D1; SPRTN; ATP11C; HERPUD1; RPS7; PDLIM5; FYTTD1; SEPT7; CDK5RAP2; TRAPPC2; PCGF6; CHCHD7; OLA1; NAA30; ARHGEF10L; BTBD1; RPS8; MSL1; MCRS1; ZNF302; CTNNBIP1; DNAJC21; AKTIP; FOXP4; SEC61G; U2AF2; CCDC66; GOSR2; CTBP1; MYPOP; SLC3A2; DCTD; ABI1; CTU2; RGMB; COA6; UBE2NL; C16orf88; RPS9; CCNC; KRIT1; SEH1L; FXR1; AGPHD1; ALG10B; C2orf68; GDPGP1; PTRHD1; SRRD; EIF2AK4; MAD1L1; EXOC7; SLTM; CXorf40B; EXOC6; SUPT20H; AKT1; CUTA; DBNL; CARS; USP21; DDX19B; ETFB; EMC6; ILK; FAM96A; TM9SF1; ZNF638; MRPL22; RPS11; FAM13A; MPG; DNAJC25; TAF9; RPS13; RFFL; SP3; TMCC1; ZNF2; MAEA; GOPC; SIRT3; ERMAP; C14orf28; ZHX1; C2orf76; CCDC58; 0S9; RAB28; VMA21; C5orf45; OPA3; RPS15; SORBS3; TPM1; CMC4; VPS13A; POLR3H; BRCC3; SERBP1; CORO1B; FPGS; VPS13C; NARG2; GCOM1; POLR2M; FAHD1; SERF2; NME1-NME2; NME2; NAE1; HAX1; RPS16; PUM1; RPS20; ZSCAN26; ZNF805; IQCB1; RPS21; GPHN; ARF1; TM2D2; CANX; KALRN; LIN52; LRRC24; ZNF688; TNRC6B; CD82; ZNF197; CBWD5; EXOC1; MINK1; YIPF5; BRMS1; ARPC4; RPS23; RPS14; ABCF1; CSNK1A1; ADAR; U2AF1; AP2M1; IRAK1; TAF5L; DUT; RAB12; ANO6; NDEL1; ARFIP1; CELF1; VRK3; FAM108B1; RPS24; RPS25; CCM2; TCAIM; KCTD21; C6orf120; PLEKHG1; GLTPD1; WDR45; ZFAT; ZNF16; METTL17; ZNF181; AP2B1; AP1G1; ARHGAP5; COX19; ZNF451; RAB24; CTNS; SRSF7; TP53BP2; PLAA; PLD3; ELP6; ERGIC1; TRMT11; CCDC90A; INF2; CRELD1; DHRS12; ZNF613; DNAJB14; DDX59; C19orf12; MRI1; YTHDC1; FDX1L; TMEM150A; TIPRL; CSNK1G3; CPT1A; KLF10; TMPO; NR2C1; UBE2V1; SLC35A2; ZNF174; ZNF207; STK24; MINOS1; ZNF226; PQBP1; LCMT1; HNRNPH2; USP48; RRM1; RPAIN; FBXO7; TMEM259; CYFIP1; FAIM; GPR155; MTERFD3; AMD1; NGRN; PAIP2; SAR1B; WIPI2; CSTF1; BABAM1; PPM1B; PHF12; RHOT1; AMZ2; MY019; ACOT9; BBS9; TRPT1; NOP2; TIAL1; UBA52; DMAP1; EIF2B4; NHP2; ITPRIPL2; RPL14; C18orf32; SRA1; UFD1L; VPS26A; BOLA3; SDHC; GTF3C2; HHLA3; EXOC4; AGAP1; FOXK1; ARL5A; GGPS1; EIF3B; THYN1; STAU1; USP14; RUFY3; GON4L; AGPAT3; SIU; BTF3; PARL; EEF1B2; GATSL3; ZNF630; NPM1; NCKAP5L; HSD17B10; REV1; DIXDC1; SLC38A10; NARF; ALG13; ATP6V1E1; NDUFAF5; ATP6V0B; NPRL3; KIAA0317; ETNK1; DNAJB2; SEC14L1; CCNL2; PICK1; DPH2; USP9X; IAHl; CREBZF; PRMT5; ZMYM5; TIRAP; YIF1B; UNC45A; CHTF8; TYW5; SNAPC3; NBPF10; SDCCAG3; DEDD; C4orf29; CDC42; OXLD1; GPX4; STRN4; FKRP; ZNF808; C19orf55; ZNF674; ZNF384; INTS6; MLLT4; TCERG1; ARL16; MAPK3; FAM133B; MOSPD3; MLH3; NRF1; PQLC2; CEP44; H2AFY; C16orf13; FAM63A; PAPD5; DCUN1D4; PRDM15; U2AF1L4; HAGH; COA3; YARS2; PHF11; ASB1; MTMR12; RUFY1; SIDT2; RHBDD2; ERAP1; EFTUD1; TMEM70; LINS; CRCP; ACP1; ZXDC; METTL21D; PPAN-P2RY11; INCENP; UEVLD; ABCE1; TROVE2; PGP; CEP63; PPP4R1; CEP170; ANKZF1; PSPC1; WHSC1; ZNF205; FAM98B; CAST; TRAPPC5; TMEM80; PSAP; SUMF2; ABHD12; ACBD5; ZNF565; GEMIN8; DLGAP4; SMIM8; ZNF706; COASY; MINA; AGAP3; SLC9A6; MAZ; NCBP2; ATPAF1; FEZ2; NSL1; SMC2; TATDN3; FRS2; EIF4G2; CHD2; ENGASE; CRTC3; SNUPN; POT1; TTC14; KDM5A; XRN1; PIGY; PARP2; NGDN; TRAK1; MFSD12; SHPRH; ZSWIM7; GTPBP10; SEC24B; STAG2; TPM3; MSMP; SMAP1; ZNF557; NET1; DPH3; MUTYH; PHACTR4; HIPK3; CLCC1; SCYL1; UBL5; TNFRSF1A; TOP2B; ACSS2; TMUB2; CLTA; UBTF; QSER1; CDC14B; ATG9A; SREK1; SENP7; SEC31A; SPPL2B; RNF214; SLC25A45; NCOR2; ZFYVE19; RBM23; POMT1; DPH5; IRF2BP2; PNKD; BCLAF1; HNRNPC; PHF16; TSEN34; PPCS; SLC39A7; MTMR14; UBXN2B; APH1A; WTH3DI; URGCP; AGAP6; ALG9; MIER1; SRSF1; FAM127B; CDC16; TMEM134; UBN1; TBCE; MED24; FAM177A1; KTN1; PAICS; TRAPPC6B; HNRNPUL2; TMTC4; FNDC3A; KIAA1191; FKTN; TMEM183B; OCIAD1; CREBBP; TAX1BP1; BCS1L; CUL4B; KIAA1147; KIAA0146; U2SURP; ZNF629; UNK; FTO; WHAMM; SNED1; BEND3; GPR108; INTS1; ZNF697; PLEKHM3; USP45; USP6NL; ZNF823; TNRC18; RGP1; TMEM223; METTL23; SETD5; BAHCC1; UNC119B; MGA; CACTIN; TMEM218; C15orf57; DNLZ; COMMD5; JMJD6; NXF1; THOC2; CPSF4; PRKDC; ZNF623; ACD; TCTN1; PIH1D2; C11orf57; ZGPAT; CHMP1A; ZNF133; CEP57L1; RABEP1; TMEM214; NAA60; TMEM219; EARS2; RB1CC1; ZBTB40; ANKRD12; STRN3; DNAAF2; WBP1L; THADA; PLOD3; DDT; DDTL; MZT2A; Cllorf83; NADKD1; CTNND1; FOXN3; MAP1LC3B2; MYSM1; C17orf89; AAMP; UQCRHL; TRAPPC13; FAM195B; TXNRD1; ACLY; RPP38; ACO2; HNRNPF; CTNNB1; LIG4; COPA; ZBTB21; ZNF621; DLG1; GRSF1; CRTC1; ZNF419; CHCHD4; DDX17; SGSM2; HTATIP2; CDK10; BAG6; USP5; TMBIM6; Clorf43; PCBP2; TMEM251; JKAMP; AKT1S1; C12orf44; RPP14; FAM89B; BET1L; MID1IP1; FAM160A2; FAM210A; INO80C; ATXN7L3; ZNF862; CCDC43; ZNF506; TINF2; COMMD7; CCNK; KAT6A; POM121C; BCAS3; ULK3; ZNF30; MTFR1L; ZNF146; FTSJD1; RPL22L1; GXYLT1; PTAR1; HIGD1A; C8orf59; EIF5AL1; REPIN1; WDR83; C4orf33; SYS1; IKBKG; C7orf25; SBNO2; IMMT; TMEM192; PDS5A; SENP6; DROSHA; C19orf60; SPATS2L; RAP1GDS1; RC3H2; KIAA0232; KDELR2; PLEKHB2; CENPN; ERLIN1; TMEM55B; MEDT; PID1; MOB4; SLC9B1; PACS2; COMMD9; CXXC1; NRD1; ACOX3; PHF21A; FOXRED2; SIKE1; HNRNPR; TTI2; PCTP; ALPK1; ZFAND5; TBC1D8; PPAPDC1B; IFT43; SNX18; ZNF160; TUBGCP5; ZNF554; OTUD4; PSMA4; RRAS2; GIGYF2; RPP30; FAM118A; PCMTD2; ACVR1; FBRS; TMEM177; RUSC1; ASH2L; CORO1C; ARMC5; ZFYVE16; FAM135A; ZNF142; MYBBP1A; ZBTB10; UBE4B; KIF13A; NUDT19; FBXO45; NUDT7; HECTD4; ZNF250; C6orf136; ADAM10; TMEM87A; SLC35E2B; MECP2; NAA16; SUPT5H; UBE2K; DDX54; TLK2; ZSCAN30; FAM208A; FPGT-TNNI3K; BRD2; NACA; ECE1; TBC1D14; FANCI; FGGY; C17orf51; SEPT9; ARHGEF7; METTL15; ENTPD6; CDC27; THUMPD3; LSM14A; C17orf85; ELK1; NBEAL1; AEBP2; IRAK4; MTRF1L; CLCN7; PAPD4; DHX36; SZRD1; JMJD7; PLA2G4B; FANCL; LIN54; KANSL3; WDR26; GDI2; ADD1; LAMP2; HCCS; CCBL1; ABCD3; MICAL3; SET; GTF3C5; TTC13; NCOA7; BSCL2; BCKDK; SMEK2; ADK; ARIH2OS; MTO1; ZBTB1; PPP6C; PARK7; BCOR; ADPRH; HDGF; CASK; OSGIN2; POLG; THTPA; AP1B1; PIGG; CFLAR; CNBP; PCID2; HMOX2; SMARCAL1; ACSF3; POLD2; AURKAIP1; AUTS2; GPBP1; LRRC8A; TMEM129; UBAP2L; CBX5; MAD2L2; MED18; ZNF84; C14orf2; TSEN15; METTL21A; ERLEC1; CRY2; CRLS1; PAN2; SPRYD7; ASAH1; ING4; NMRK1; PEX26; MFN2; ATXN3; TMEM14B; STXBP5; SPG21; CEACAM19; AP4S1; RWDD3; TFRC; ORMDL1; VPS53; UBP1; NUDCD1; KCTD6; VGLL4; ZNF717; SLC39A13; DIS3; GNE; TPRN; LYRM1; LACC1; AP1AR; SMARCAD1; PSMG4; MAPKBP1; USP5; NUDT22; REPS1; LUZP6; DCAKD; SMARCA4; SRRT; GTPBP3; TOMM40; MARK3; INPP1; ENTPD4; NSDHL; TEX264; DNAJC2; KRBOX4; SYCE1L; KIAA1841; AES; GSPT1; ATP6V0A1; ZNF680; CLK3; ZNF562; SHC1; TBCEL; ATF7; MYO9B; EPN1; KARS; COL4A3BP; HSPBP1; FAM108A1; RFC5; SMARCC2; SPTAN1; SRP9; HRAS; SSFA2; HAUS2; THAP5; VRK2; ZNF195; AP1M1; SPAG9; CALU; EIF4E; STYX; C14orf93; LSM5; PSMB5; CCDC149; DNMT1; RTCA; AIFM1; CAB39; PPIP5K1; PWWP2A; SUGT1; ZNF720; TGFBR1; MEF2A; C7orf73; PLCD1; SUN1; HYOU1; FAM58A; PTPN12; SATB1; CIZ1; ATG10; ZCCHC9; SAP30L; ACP2; TMEM106B; EIF2AK1; PSMG3; MAP4; LRRFIP2; NT5C2; CCNJ; TBC1D5; IQSEC1; ZDHHC4; C7orf50; TBCCD1; CDV3; AZI2; C3orf58; GSE1; PARN; HS2ST1; TOMM6; TRMT10A; DERL1; FAM204A; DEK; ARFRP1; IPO11; CCDC152; FIP1L1; ELMOD3; PDHX; MFAP3; DCTN1; MAPK9; FAM160B1; FNDC3B; CRELD2; DNAJA3; NEDD1; ZNF397; ZDHHC3; AGFG1; FKBP2; GIT2; TAF12; LDHA; RBBP4; MKNK1; HDHD1; C12orf73; SMIM13; C5orf24; GDAP2; RPS27A; PPP1R21; PIP5K1A; INPP5K; DCTN4; FAM53C; PTPRK; EEF1E1; EIF2AK2; XPR1; MSRA; ATL2; C8orf40; VDAC3; YWHAZ; HMBOX1; NEIL2; ECD; RPN2; SPATA2; FDPS; RNF185; PHPT1; METTL20; SLC46A3; KIAA1432; MADD; URM1; UCK1; NDUFB11; RUSC2; ABL2; ATG7; PUF60; TRMT1; NIF3L1; CPSF7; PTGES3L-AARSD1; TMUB1; TPRA1; R3HCC1; FBXO28; FAM178A; RPL28; RPS6KC1; CMPK1; ATF6B; ZNF507; OTUD5; FASTKD2; TNPO2; FZR1; ISOC2; CCDC124; RCOR3; SEC13; SGMS2; ATXN7L3B; AKIRIN1; ANP32E; CISD3; ACAD10; APOL1; LYSMD1; TLK1; GPR107; LANCL1; LRRFIP1; MCTS1; ANAPC5; MEMO1; POLR1B; ANAPC7; ILF3; ATXN1L; BCAP31; TTLL11; CNST; TBL1X; TRAF3IP1; PRKRA; DAXX; ATP13A2; TP53BP1; RAB11FIP3; CLASP1; APLP2; RNASEH2B; ARCN1; SMC6; EMC8; MGRN1; LMAN2L; ARFGAP3; SQSTM1; GTF2H1; TXNL4B; DMTF1; THOC6; PPP3CB; ALG5; PNPLA4; CTIF; CD164; AIMP1; MORF4L2; MGEA5; EDC3; SPNS1; DKC1; ECSIT; C6orf203; INTS12; FLYWCH2; MON1A; SLC35B3; ADCK1; RPUSD3; ADCK4; RRNAD1; RAD51D; ZNF669; NFYC; ITPK1; CLP1; KIAA0141; EFTUD2; ULK2; EHBP1; TGFBRAP1; GHDC; TNRC6C; FBRSL1; SAR1A; HNRPLL; ATG13; CHID1; ERI2; C1orf122; IL11RA; C17orf49; EYS; APIS; DAGLB; MPC2; GSTK1; DIS3L; EIF5A; ZNF438; CTDNEP1; SLC25A39; PPHLN1; TPCN1; ZBTB14; MAPRE2; NFRKB; TMEM106C; TCHP; WIBG; COPS2; BSDC1; C12orf65; TRAFD1; LOC729020; C15orf61; PSMA1; LEMD2; TMEM30A; C2orf74; TBC1D7; CDYL; TCTN3; PTPMT1; BANF1; WRAP53; AMFR; AGAP5; CTPS2; TMX2; NAT10; COPB1; UBAC2; DET1; DNAJC7; CD58; DENND4A; PHB2; IMPAl; SMCR7; C11orf95; MYL12B; DTWD1; NFKBIL1; MTHFD2L; ZNF814; CCDC85C; ITGAV; COG2; GPN1; SLC44A2; USP27X; COG6; ZNF619; SKIL; RRP12; MKRN1; AKD1; RELA; VPS37A; HBS1L; INTS9; DOHH; PRMT3; KIAA1671; LAMTOR2; SLC35C1; FAM185A; NGLY1; ETV3; DSN1; ZNF566; ZNF576; KDM8; IPP; MKLN1; CBWD1; SIN3A; ABHD11; ZNF652; OXSM; TSEN2; TEF; NONO; NFE2L2; SETDB1; TMEM205; C4orf52; PGAP2; SCAF4; SPECC1L; EHMT1; TCP11L1; RBM17; ZDHHC7; KIAA0226; GLG1; SAEl; HOMER3; XPC; MEF2BNB; SH2B1; MTFR1; SARS2; SCAPER; SLC12A4; RDH13; TJAP1; FCHO2; HSDL1; TDRD3; RPAP3; FAN1; PARP9; DIP2A; GSK3B; MOGS; TATDN1; ZNF414; ZNF407; TBC1D15; WRB; PIP4K2C; TCF7L2; SRP54; LEPRE1; Clorf86; PQLC1; KDM3A; KDM4C; RBM19; KDM5C; SLC25A5; ANXA4; SCOC; ANXA6; ANXA7; ANXA11; MTHFSD; BIVM; BOD1; SYNCRIP; PLBD2; BUD13; RIOK2; CANT1; MPND; EBNA1BP2; EVI5L; EPS15; TXNDC16; ACOT13; C15orf40; RNF170; SPG11; SETD6; SETDB2; TRAPPC9; POLR3B; NUDT2; ARMC10; CHFR; NPTN; NDFIP2; JMJD4; WDR25; COG5; TNIP2; RBM34; TEX10; DUS3L; PPP2R5C; CLK1; PDCD6IP; TMEM189; RBMXL1; COX11; TYW3; RPTOR; HTATSF1; EWSR1; FBXL17; RAB2B; ZSCAN12; ZNF580; MYEOV2; TBCK; ZNF746; DCAF11; DCAF4; GTF2I; WDR81; KCNMB3; C10orf2; COPS7A; CHAMP1; PPP6R3; GPR75-ASB3; PLIN3; DHX16; Clorf27; WDR46; TRAF3IP2; FLNB; BRD8; THAP4; GPN3; STAU2; MTF2; TMED7-TICAM2; EIF4ENIF1; C16orf52; ASXL1; ENDOV; ZFHX3; BCAT2; SLC25A26; RBMX; PET117; ACIN1; DCAF17; SMIM12; LYRM4; TMEM41B; DTYMK; TMEM14C; NFKB1; SLC25A11; CD320; MKS1; DAG1; STARD3; IDE; ELAC2; BIRC2; ECI2; ERCC1; NDUFV1; TADA2A; PNPLA6; RBM28; LCORL; NDUFS2; UTP14A; CEP120; C22orf39; FHIT; MTIF3; HAUS4; DHX40; PIGX; SHMT2; HDAC8; WDR13; MPP1; SLC16A1; EIF2B3; FAM122B; TRAPPC1; AFF1; FAM104B; XIAP; RBM6; XPNPEP1; RAB35; RHBDD1; LEMD3; ATXN10; LPP; VARS2; SMYD3; TMED5; NSMCE4A; ATP5SL; LHPP; ANKRD50; TIMM17B; TRMT2B; TBC1D17; NDUFB4; ME2; NSUN5; CULT; SLC35A1; TSPAN3; ARMCX5; CNDP2; TMEM48; IFT46; TXLNG; TMEM135; FAM21C; SCO2; STIM2; TJP2; CDK16; CDK17; ATAD3A; PGAM5; CXorf56; CHD8; FUS; LPPR2; SRGAP2; LAS1L; ZNHIT6; MIB2; GPR137; PIN4; LCOR; MFSD5; ATRAID; ZFAND1; LARP4; RBM41; SMPD4; UBXN6; FAM3A; STRBP; PET100; CAMTA2; UBAP1; MCFD2; TRIQK; PAPD7; PPARD; FGFR10P2; VPRBP; NUDT16; CXorf40A; KXD1; RBFA; SETD9; MASTL; VANGL1; BAG1; RAB3GAP1; RRM2B; GOLGA3; MCPH1; NEO1; TECPR2; TK2; RAB40C; ZNF668; ZNF347; ZNF764; ZNF641; TSFM; PPARGC1B; SLC38A6; GGA3; GOLGA4; SEC23B; DPY19L3; ZNF555; YTHDF2; TFCP2; AAAS; CRBN; NKRF; MRRF; DGCR2; BANP; BRD7; SMG7; POLL; NCOA3; PCBP4; ZBED6; ARL13B; RABEPK; SAMD8; ARL1; ABHD16A; PPP2R2A; SUCLG2; CINP; RIF1; IFT27; KLF11; RANGRF; SRPR; SYCP3; MNAT1; ECI1; SF1; ZC4H2; ZFX; SYNJ2; MINPP1; SUFU; ATP6AP1; ATR; HADH; TIPARP; PIGT; CTTN; ZBTB33; PAFAH1B2; ZNF408; UHMK1; VDAC2; PEX11B; ESYT1; TMLHE; UBR2; CD99L2; GNL3L; PRMT7; KLHDC4; FLAD1; FBXL20; WDR44; PACSIN2; UQCC; NDUFS5; WNK1; NDUFC1; KIAA0430; RNF4; NCAPH2; NDUFA2; ZDHHC8; ACOX1; ZCCHC6; ZNF75D; FMR1; ARHGDIA; NIT1; MYNN; PFDN6; BAK1; DNAJC19; C1D; ATG16L1; FBXO11; DGCR8; TAF6; NCOR1; IKBKB; ZNF317; NCK1; DHX35; SMAD7; MRPS35; ORC4; HYI; FAM193B; ZMYM2; YAF2; IL6ST; SRSF11; SLC33A1; IPO8; ARPC1A; BCL2L1; GSTO1; SRSF10; CTCF; TNPO3; PSMD1; SIRT5; EML2; MSL3; RBBP5; SIRT6; SIRT2; TMEM127; VIPAS39; C9orf3; MRPS18A; NUP62; EXD2; DIDO1; NDUFA11; UCKL1; PPP2R4; DDX3X; NSUN2; KANSL1; LIMS1; SLC1A4; REST; TTC27; SLC30A6; CHMP3; FAM65A; SCRN3; NEK4; FBXL5; ENY2; TUBD1; DHRS4L2; PEX19; POGZ; EIF4G1; MATR3; MEPCE; MR1; PPIE; TMEM184B; ANKRD28; PTP4A2; COG4; NASP; CCDC107; YIPF6; DENND1B; APTX; SERPINB6; USB1; RAB9A; SRSF2; MICU1; CHMP5; CLINT1; CAMTA1; DICER1; SEPHS1; ZNF865; TOPORS; MLLT10; VAPB; THAP3; HSDL2; ANKHD1; ZFP91; MLL; GCLC; IRF3; BCL7B; ORC3; GABPA; MCL1; HIRIP3; ARNT; OXR1; ATP6VOC; JMJD7-PLA2G4B; ARHGEF12; LEPROT; RBBP7; PI4 KB; CUL2; POU2F1; ARPC4-TTLL3; ASCC1; EIF4G3; MSANTD3; MSANTD3-TMEFF1; RBM14; RBM12; CCT2; RBM4; RBM14-RBM4; CPNE1; CAPN1; ATP5J2-PTCD1; YY1AP1; ATP6V1F; ABCC10; RNF103; RNF103-CHMP3; TMEM110-MUSTN1; NFS1; DCTN5; CDIP1; C15orf38-AP3S2; NT5C1B-RDH14; TBC1D24; TRIM39-RPP21; RPP21; COPS3; TANK; AMMECR1L; KAT7; USP19; PSMC5; MLST8; CCNH; ARMC6; TBC1D23; AK2; GPANK1; TOR1AIP2; UCHL5; CABIN1; LRBA; UIMC1; CNOT2; BLOC1S5; FPGT; RPL17-C18orf32; GBF1; RNF145; NEK1; TRAF3; NIP7; PDCD2; ISY1; ZSCAN9; C20orf24; TGIF2-C20orf24; SUN2; PTK2; PMF1; PMF1-BGLAP; SLC4A2; DHX33; PPP2R5A; PSMA5; CPD; POC1B; PSMB2; INTS7; GGCT; MDP1; NEDD8-MDP1; SMURF1; DAP3; AK3; BCL2L2-PABPN1; KIF16B; MARK4; GLRX3; B4GALT3; HYPK; PDK2; PGM3; SIAE; SESN1; DOPEY1; SH3GL1; NDUFB5; UQCRB; NDUFB6; GCFC2; SAFB; HMGN3; RNF14; RNF7; ZNF778; GORASP2; ZNF513; C18orf21; EIF2D; CORO7-PAM16; PIGO; RBM15; PLRG1; SEC22C; ASB3; ASB6; AKR1A1; TRMT1L; PRDX1; C10orf137; ZMYND11; RPS10-NUDT3; UBE2E1; HSPE1-MOB4; UBE2G2; UBE2H; CTDP1; CUX1; SYNJ2BP-COX16; PIGV; CHURC1-FNTB; WBSCR22; MTA1; NDUFC2-KCTD14; IL17RC; NDUFC2; COMMD3-BMI1; CHURC1; UBE4A; COX16; PPT2; MBD1; SPHK2; MDM4; ZHX1-C8ORF76; SRP19; ZNF670; SCARB2; PPPSC; ZNF664; PRPS1; BIVM-ERCC5; CCPG1; PSMC2; RBAK; RBM10; EIF4A1; RBAK-LOC389458; KIFAP3; RFC1; ZNF587; LIPT1; ANO10; TNFAIP8L2-SCNM1; SCNM1; TCEB1; URGCP-MRPS24; NPEPL1; BAG4; ISY1-RAB43; BNIP1; TTF1; KLF9; USMG5; MAVS; CAPZB; POLR1D; CHTOP; AKIP1; SH3GLB1; IGSF8; PRKAG1; NSFL1C; GTF3C3; ARID4B; MAP2K5; KAT5; RAB11A; TGOLN2; STRADB; FAM115A; DHPS; HNRPDL; PTPN2; M6PR; RNF40; PRMT1; ATRN; BACE1; VWA9; BZW1; C1QBP; ZNF48; CAMK2D; CASP6; CASP7; CASP9; CCNT1; CCNT2; PITRM1; ATAD2B; ODF2; ANAPC13; TWF1; WDR20; PIK3R1; EIF1AD; ZSWIM8; MIF4GD; MFSD11; NCOA6; ANAPC16; MAP4K4; RIN2; TMEM147; RBM39; RAB2A; AHCYL1; LOC100289561; ZNF691; TRIM26; BRF1; NUP93; ZNF322; ZNF790; DEF8; RNF41; ARFGAP2; AP2A2; RNF146; ARFIP2; ELP2; CARKD; ZBTB17; ZKSCAN3; PPP6R2; AKAP1; MPPE1; ASCC2; ZFAND6; EIF3L; ZNF410; SNX1; AKT2; PLD2; NFKBIB; PDE8A; TAF1C; PIM1; INPP5F; HIP1; RANBP6; PES1; NARS2; TIGD6; HINFP; NUB1; CLCN3; GLRX2; CLEC16A; PDIK1L; MTMR2; CD2BP2; GFOD2; LETMD1; RAB6A; SETMAR; LAMTOR3; RGL2; C7orf49; POMGNT1; BTF3L4; CEP57; SMUG1; CHST12; TOB1; TRA2B; TPD52L2; HDLBP; PRPSAP2; PPP3CC; KIAA0586; APEX1; HBP1; TRRAP; C7orf55-LUC7L2; LUC7L2; IMMP2L; CHMP2B; STX5; GFPT1; RAD23B; TMEM126A; FOXP1; DLST; PRPF4; TXN; PPP1CC; SEL1L; CTAGE5; ASAP1; TRIM3; NUDT9; SP1; USP4; ASPSCR1; APPL2; SLC30A5; PAPOLA; RAB5B; RAB5C; TAOK2; PCMT1; USP15; AP4E1; LSM4; GEMIN5; SEC24A; CEBPG; NT5C; TNIP1; URI1; ACSS1; BBS4; CDC5L; RPL15; ZNF444; SLC52A2; GMDS; AP4B1; YME1L1; UXS1; MED27; TBC1D1; CYB5D2; CREB3L4; PNPLA8; PSMC3IP; PIK3CB; ANKRD26; C9orf72; ATF2; NAA10; TRIM65; CERS6; ARL8A; CSE1L; TMCO1; ZNF620; ANKRD11; SNX12; ARAF; ETS2; STK3; PTGES2; CHD1L; UBE2L3; MCMBP; LRRC39; NOL8; ELOVL1; SLMO2; KDM2A; LRRC42; RAB18; CPSF3L; KAT6B; WDR92; GOLGB1; MAN2C1; SSBP1; C9orf69; SLC25A1; NOP16; PCGF5; MPP5; PPFIBP2; RPL10; Clorf85; TUBGCP2; R3HCC1L; NR1H2; FAM193A; DPP3; STOML1; KIAA0391; CSNK2A3; PRDM11; ANAPC10; CCT4; USP39; CNOT10; TMEM161A; GAPDH; RIT1; PAF1; SMG6; LOC100862671; POLD1; BTRC; RNF34; SRI; DDX21; CLCN6; CCDC51; FBXW7; NDUFB3; COX14; ITCH; DDX56; POM121; DDX6; CUL3; DIS3L2; HNRNPH1; SCFD1; ABCG2; CD63; TRMT2A; CCDC132; ANKFY1; COPS4; SERINC4; POLR3E; HARS; MIS12; NDUFA12; SPATA20; IDH3B; FAM173B; SMS; TARS; FBX018; FASTK; CDK8; WDR4; ZNF155; SLC9A8; RDX; SRP68; CDK9; CALCOCO2; NOL10; PSMD9; TSN; SFSWAP; DCTN2; LPIN1; AARSD1; ADAM15; NSRP1; PDPK1; AP3D1; TBRG4; BRE; MORF4L1; CNOT1; MZF1; LARP7; ARMC8; PSME3; SNX17; PEMT; PDCD6; EIF3C; TOR1AIP1; UBOXS; FAM189B; ITPA; SRP72; CCDC61; ARSG; ING1; IFT20; AMBRA1; PAAF1; ILF2; EIF6; SLC12A9; ZNF839; CLOCK; SLIRP; HSD11B1L; SHOC2; CHD1; TMEM254; ANKRD46; FAM73A; RXRB; MAP4K3; PSMD5; CDK2AP1; UBE3B; WWP2; MCM3; PPP2R5D; PSMB6; PSMD11; CAMKK2; TAF11; RPL13A; LATS1; DAAM1; MED23; STOM; RNF111; WTAP; MED4; JOSD2; MARCH6; MCU; ARHGAP12; BCL2L13; NTAN1; STRIP1; TFAM; MEAF6; HAUS6; TRAPPC6A; TRAPPC3; UCHL3; NOSIP; IST1; ZFAND2B; MAX; VPS72; PCED1A; RAP2C; FAM173A; TTC19; EMC1; C21orf2; PEX11A; DNAJC10; LOC100129361; PPME1; HERC3; STX10; PPP1R12C; RQCD1; ZNF138; MTCH1; NSA2; LOC441155; PYCR2; SLC35A3; ABCB7; MKRN2; FBXO38; COPZ1; APEX2; AP3B1; PSMD6; DYNC1I2; MED21; DCLRE1A; PRELID1; RSRC1; RCN2; IKZF5; ZNF700; CDK2AP2; RRAGC; GTF2H3; AAR2; CUEDC1; KHDRBS1; AAGAB; TARS2; SEC11A; CEP164; RMND1; MEGF8; SLC39A1; HSP90AB1; STK25; PUS3; RAB4A; DOCK7; EPC1; LRRC14; RPS6KB1; TRAP1; C16orf91; MRFAP1; SHISA5; ABHD10; QARS; USP10; STX4; CHD4; WDTC1; RGS3; MBD4; PPIP5K2; PRKAR1A; NISCH; PPP1R3E; YOD1; C18orf8; USF1; ESF1; UNKL; SEC16A; KPNB1; ELF2; LONP1; CHUK; CIRBP; TBCB; AP1S1; AP3S1; CLNS1A; CLPTM1; CREBL2; MAPK14; CSNK1G2; CSNK2B; CSTF3; CTSO; CTSZ; DAD1; DGKQ; DARS; DHX9; DHX15; DECR1; DNASE2; DYNC1H1; DPAGT1; DPH1; DRG2; DYRK1A; ECH1; EEF1G; EIF2B1; EIF2S3; EIF4B; ELAVL1; ENO1; EP300; FBL; EXTL3; XRCC6; BLOC1S1; GDI1; GTF2B; GTF2H4; GTF3C1; HDAC2; HSBP1; DNAJA1; NDST1; ICT1; IL13RA1; ING2; INPPL1; EIF3E; AARS; ACVR2A; PARP1; AKR1B1; APEH; TRIM23; ARF4; ARF5; ARF6; RHOA; ARVCF; ATF4; ATPSB; ATP5F1; ATP6V1C1; ATPSO; AUH; POLR3D; BPGM; BSG; CAT; CBFB; CDK7; CENPB; CENPC1; CLTB; SLC31A1; COX4I1; COXSB; COX6B1; COX7A2; COX7C; CSNK1D; CSNK2A1; CTNNA1; CTPS1; CTSB; CTSD; CYC1; DBT; DDB1; DLAT; DR1; DUSP7; E2F4; EEF2; EIF5; ELK4; STX2; ESD; ETV6; EYA3; FAU; FKBP3; FKBP4; FNTA; FNTB; FTH1; KDSR; GAB1; GABPB1; GARS; GCLM; GNAQ; GNB1; GNS; GOLGA1; GOT2; GTF2E2; GTF2F1; GTF3A; H2AFX; H2AFZ; HTT; HIVEP1; HMGB1; HNRNPA1; HNRNPA2B1; HNRNPK; HSPA4; HSPD1; HSPE1; IARS; ID2; ID3; AC01; IRF2; ITGAE; ITGB1; ITPR2; JAK1; KPNA1; KPNA3; KPNA4; TNPO1; IPO5; LIG3; LRP1; LRP3; LRP6; LRPAP1; MAGOH; MAN2A1; CD46; MDM2; MAP3K3; MGAT2; MGMT; MIF; MAP3K11; MPI; MPV17; MSH3; MAP3K10; MTAP; MTRR; MTX1; MVD; NUBP1; NBN; NCBP1; NDUFA4; NDUFA6; NDUFS4; NDUFS8; NFX1; NFYA; NME3; NRAS; NTHL1; NUP88; NVL; TBC1D25; OAZ2; ODC1; OGG1; ORC5; OSBP; PEBP1; FURIN; PAK2; PBX2; PCNA; PDE6D; PERI; PEX10; PEX13; PFDN1; PFDN4; PFDN5; PFKL; PHB; SLC25A3; PHF1; PIGA; PIGC; PIGF; PIK3C2A; PIK3C3; PI4KA; PMM1; PNN; POLA2; POLR2E; POLR2G; PPAT; PPP1R7; PPP1R8; PPP1R10; PPP2CA; PPP4C; PREP; PRKACA; PRKCI; MAPK1; MAPK6; MAPK7; MAPK8; MAP2K1; MAP2K3; PRPSAP1; PSMA2; PSMA3; PSMA6; PSMA7; PSMB1; PSMB3; PSMB4; PSMB7; PSMC1; PSMC3; PSMC6; PSMD2; PSMD3; PSMD4; PSMD7; PSMD8; PSMD10; PSMD12; PSMD13; PSME2; PTBP1; PTPN1; PTPN11; PTPRA; RAD1; RAD17; RAD51C; RAF1; RALB; RANBP1; RANGAP1; RARS; RASA1; ARID4A; RCN1; NELFE; RECQL; UPF1; REV3L; RFC2; RFC4; RFNG; RFX1; RGS12; RING1; RNASEH1; RNH1; RORA; RPA1; RPA2; RPA3; MRPL12; RPN1; RXRA; SBF1; ATXN2; SDHB; SDHD; MAP2K4; SRSF3; SGTA; SKI; SMARCA2; SMARCC1; SMARCD1; SMARCE1; SNAPC1; SNAPC4; SNRNP70; SNRPB; SNRPB2; SNRPC; SNRPE; SNRPF; SNRPG; SNX2; SP2; UAP1; SPG7; SPTBN1; SRM; SRP14; SRPK1; SSB; SSR1; SSR2; SSRP1; STAT3; STIM1; STRN; SUPT4H1; SUPT6H; SUPV3L1; SURF1; SUV39H1; ADAM17; TAF2; TAF4; MAP3K7; TAPBP; TBCC; TCEB3; TCF12; TDG; TERF1; THOP1; SEC62; TRAPPC10; TOP1; TPP2; TPR; TPT1; NR2C2; TSPYL1; TSSC1; TSTA3; TTC1; TUFM; HIRA; TYK2; UBA1; UBE2A; UBE2B; UBE2D2; UBE2D3; UBE2G1; UBE2I; UBE2N; UBE2V2; UNG; UQCRC1; UQCRC2; USF2; UVRAG; VBP1; VDAC1; XPO1; XRCC4; YY1; YWHAB; ZNF7; ZNF35; ZNF45; ZNF76; ZNF91; ZNF131; ZNF134; ZKSCAN1; ZNF140; ZNF143; ZNF189; ZNF202; USP7; STAM; CUL5; MLL2; TAF15; NRIP1; TMEM187; AXIN1; HIST1H2BC; PIP4K2B; ULK1; EEA1; ANXA9; STX7; VAPA; ZNF282; DUSP11; CUL1; TTF2; SMARCA5; OFD1; PPM1D; RANBP3; PPFIA1; PARG; NDST2; IKBKAP; HAT1; DGKE; CAMK1; AGPS; BLZF1; MAPKAPK5; PRPF18; DEGS1; DENR; YARS; RRP1; KHSRP; AKR7A2; NOP14; RUVBL1; USO1; CDK13; RFXANK; SSNA1; NCOA1; TNKS; EIF3A; EIF3D; EIF3F; EIF3G; EIF3H; EIF3I; EIF3J; BECN1; MRPL40; B4GALT4; MBTPS1; EDF1; CTSF; SNX4; SNX3; EED; RNMT; RNGTT; GPAA1; RIPK1; CRADD; TNFSF12; ADAMS; CDS2; RIPK2; FADD; SNAP23; NAPG; NAPA; MTMR1; RIOK3; TNFRSF10B; DYRK4; SUCLG1; SUCLA2; CREG1; TRIM24; DPM1; DCAF5; DPM2; SAP30; CES2; TMEM11; HDAC3; KAT2B; SGPL1; FUBP1; ZNF259; MCM3AP; EIF2B5; EIF2S2; CPNE3; BUD31; PRPF4B; TIMELESS; HERC1; MBD3; MBD2; ST13; FUBP3; TOP3B; WASL; ATP6V0E1; SLC25A14; RPS6KB2; RNF8; UBA3; UBE2M; BTAF1; AIP; CLK2; RHOB; ATIC; ATOX1; BYSL; CCNG1; CDKN1B; AP2S1; COX8A; CRY1; CS; TIMM8A; DUSP3; ECHS1; EIF2S1; EIF4EBP2; FDX1; FEN1; GMFB; GPS1; GTF2F2; HSPA9; IDH3G; IREB2; NDUFB7; NINJ1; OAZ1; PRKAR2A; RAB1A; RAB5A; SDHA; SNRPD3; TARBP2; UXT; PIGQ; FIBP; EBAG9; RAB11B; UBE2L6; MFHAS1; CYTH2; MED14; SOCS6; ZNF235; TRIP12; TRIP11; JMJD1C; MED17; MED20; PIGL; PMPCB; GTPBP1; NFE2L3; MTRF1; ACTL6A; ACVR1B; ARHGAP1; ARL3; ASNA1; BAD; BCL9; BNIP2; BPHL; BRAF; PTTGlIP; CAD; CALR; CASP3; CD81; CDC34; COX6C; COX15; CREB1; CTBS; DDX5; DDX10; DFFA; RCAN1; DVL2; DVL3; E4F1; PHC2; ENDOG; ENSA; EPRS; ERH; ESRRA; ACSL3; ACSL4; BPTF; FARSA; FDFT1; FLOT2; FRG1; GALNT2; GOLGA2; GPS2; ARHGAP35; GTF2A2; HNRNPAB; HNRNPU; HUS1; IDI1; FOXK2; MGST3; MOCS2; NARS; NDUFA1; NDUFA3; NDUFA10; NDUFB1; NDUFB2; NDUFB10; NDUFS3; NDUFS6; NFATC3; YBX1; PARK2; PET112; PEX14; PIGH; PSPH; RABGGTA; RABGGTB; RPS6KA3; SCO1; SNRPA; SNRPD2; SREBF2; TAF1; TBCA; TOP3A; TRAF6; TTC4; RAB7A; PRRC2A; DDX39B; PABPN1; C21orf33; BAP1; CDC23; HERC2; PIAS2; MTMR6; MTMR4; ATP6V0D1; PRPF3; FAM50A; RRP9; PRKRIR; ATG12; PDCD5; HGS; NEMF; PCSK7; COX7A2L; SCAF11; AP4M1; ZW10; ETF1; MTA2; NOLC1; MAPKAPK2; ITGB1BP1; COPB2; ZNHIT3; MED1; B4GALT5; CNOT8; VAMP3; SNAP29; TXNL1; PPIG; KIF3B; TM9SF2; CIAO1; POLR2D; HS6ST1; NMT2; PEX16; SNRNP40; DDX23; SYMPK; EIF2AK3; SH3BP5; EIF4E2; ATG5; ROCK2; STX8; PIGB; CLTC; FXR2; MPDU1; TMEM59; CIR1; APBA3; ATP6V1G1; SPAG7; MRPL33; SEC22B; PRDX6; VPS9D1; SEC24C; ACTN4; MRPL49; DDX1; DHX8; MTOR; KRAS; MARS; MYO1E; NDUFA5; NDUFA7; NDUFA9; NDUFAB1; NDUFB8; NDUFB9; NUCB2; OXA1L; PCYT1A; PFN1; PGGT1B; PIK3R2; POLR2K; POLRMT; PPID; PRCP; PWP2; ABCD4; SFPQ; SIAH2; TLE1; TRIM25; NUP214; ZRSR2; SLC27A4; ZMYM4; RBM8A; OXSR1; WDR1; GOLGA5; MVP; THRAP3; MED12; MED13; NUP153; CCS; DOPEY2; THOC1; SART1; ABL1; ATF1; BMI1; CHKB; CRK; CRKL; DDOST; ERCC4; GAK; GFER; GLUD1; GNB2; RAPGEF1; PDIA3; HCFC1; HINT1; ZBTB48; HSPA5; JUND; SMAD4; NCL; NFIL3; NKTR; NUP98; PDCL; PHF2; RALA; ROCK1; SLC20A1; STAT2; YES1; CCDC6; MLF2; SMC3; ZRANB2; MED6; ACOT8; GNPDA1; MED16; PIGK; RANBP9; UBA2; CFL1; DMXL1; DOM3Z; GTF2E1; HSF1; DNAJC4; IDH3A; IFI35; IFNGR2; INPP5A; INPP5B; LAMP1; LMAN1; ALDH6A1; MRE11A; RBL2; RHEB; SRSF4; SOLH; SOS1; TAF13; TARBP1; ZNF354A; TCF20; TERF2; NELFA; EVI5; REEP5; TAF1B; SOX13; FARSB; ABCC5; DNM1L; ABCF2; COX17; SCAMP2; SCAMP3; ERAL1; TSSC4; PDCD7; GIPC1; ARPC3; ACTR3; PPIF; CTDSP2; ARPC2; RAD50; ACTR1B; ACTR1A; ZNF263; PDIA6; ARIH1; NAMPT; AKAP9; G3BP1; CEBPZ; TRIM28; ATP6AP2; LPCAT3; RCL1; CNIH; RBM5; LHFPL2; ALYREF; TXNDC9; MPHOSPH10; NME6; NUTF2; USPL1; EIF1; FLOT1; PSMD14; PRDX2; PRKD3; SLC35B1; DCAF7; AP3S2; MRPS31; POP7; SRRM1; STAM2; SF3B4; ZMPSTE24; AKAP8; PURA; STUB1; STAG1; SIGMAR1; CWC27; SAP18; SMNDC1; BCAS2; EIF1B; DNAJA2; APC2; KATNB1; ACAT2; CAPRIN1; NBR1; MCMI; MDH2; MAP3K4; MFAP1; MIPEP; MLLT1; MTHFD1; NAB1; HNRNPM; NAP1L4; PRCC; RNF6; TSPAN31; TBCD; TSNAX; UQCRFS1; UQCRH; CLPP; LAGE3; ARID1A; ALKBH1; CDC123; H1FX; PCNT; CDC42BPB; HDAC6; SNAPC5; DSCR3; SMYD5; RRAGB; AGFG2; TUBA1B; IK; IRF9; BPNT1; PIAS3; LUC7L3; TAB1; MAN2A2; TMEM50B; CAPZA2; DYNC1LI2; NEDD8; NFYB; NUCB1; NUMA1; ORC2; PA2G4; PCBP1; PCM1; PIK3CA; PIN1; PITPNA; POLE; POLR2H; POLR2I; POLR2J; PPP2R5B; PPP2R5E; PRKAA1; PRKAB1; PKN2; DNAJC3; PSME1; RAD21; RANBP2; DPF2; SRSF6; ITSN2; TAF10; TESK1; TSG101; VARS; XRCC1; ZKSCAN8; SHFM1; ANP32A; SMC1A; NPEPPS; PCGF3; CDIPT; PGRMC2; ARIH2; TUBGCP3; CFDP1; RAN; TIMM23; LYPLA1; EMG1; TIMM17A; ZER1; HMG20B; MERTK; SLC30A9; PIBF1; PPIH; ZNHIT1; TIMM44; ZBTB18; TADA3; UBE2E3; EIF3M; SEC23A; CREB3; LRRC41; VTI1B; ENOX2; APPBP2; CIB1; CHERP; IPO7; NOP56; SSSCA1; RNASEH2A; ANP32B; LAMTOR5; AGPAT1; SPTLC1; ARFGEF2; ARFGEF1; RABAC1; SLUT; SIVA1; MRPL28; NPC2; TXNRD2; DRAP1; DNPH1; PRPF8; PAIP1; TBL3; MXD4; HEXIM1; RBCK1; STAMBP; POLR3F; POLR3C; IVNS1ABP; TAF6L; ATP5L; GNAI3; LGALS8; POLH; PSMC4; TRIM27; RSC1A1; SARS; DYNLT1; DYNLT3; TFE3; SLBP; YEATS4; ELL; NCOA2; SPHAR; EXO05; NPRL2; MTX2; YKT6; PMVK; FARS2; CGRRF1; RRAGA; DCTN6; GNA13; MAP4K5; GMEB1; CCT8; POLD3; HSPA8; SLC12A7; NUDC; PTGES3; MAP3K2; ZBTB6; POP4; VAMPS; ZNF460; RPP40; SDCCAG8; CLPX; SRCAP; JTB; MAN1A2; TXNL4A; NUDT3; GLO1; EHMT2; COPSE; RNPS1; SUB1; SMPDL3A; DIAPH2; PSKH1; SURF6; SYPL1; TALDO1; TCEA1; YWHAE; IFRD2; LZTR1; LMO4; DDX18; QKI; ZFPL1; WDR3; MALT1; RALBP1; PRDX3; AFG3L2; KDELR1; SF3A3; HNRNPA0; SEC61B; SERINC3; PNRC1; PSMF1; TMED2; STIP1; CKAP4; YWHAQ; TMED10; ASCC3; UQCR11; COPS6; GCN1L1; COPS5; METAP2; SF3B2; ILVBL; SNRNP27; TMED1; LIAS; CALM1; MYO9A; PPA2; RAC1; RBBP6; RNF5; RPE; SDF2; ST3GAL2; SKIV2L; SKP1; SUMO3; SNRPD1; SOS2; ZNF33A; ZNF33B; ZNF12; ZNF17; ZNF22; ZNF24; ZNF28; ZBTB25; RNF113A; NPM3; SLC35D2; ADRM1; NUDT21; CPSF6; RTN4; DDX52; WWP1; CYB561D2; TMEM115; DUSP14; TOPBP1; RER1; HNRNPUL1; KRR1; FAF1; POLR3A; CLASRP; KPTN; PWP1; CDC37; FICD; LSM6; ATPSI; RPL10A; UBL3; SSR3; TCEB2; TEP1; TFDP1; TMF1; TRIO; UTRN; VCP; ZNF41; VEZF1; ZNF175; ZXDA; ZXDB; SLMAP; ZMYM6; TESK2; NUP50; C14orf1; STRAP; CEP250; WBP4; ABCB8; SEC23IP; SUPT16H; POLI; PROSC; AKAP10; MRPL3; RPL35; PRAF2; SEC63; HPS5; RNF139; DCTN3; XPOT; CHP1; PXMP4; DUSP12; SNF8; ATXN2L; SYNRG; PNKP; B4GALT7; VPS45; LYPLA2; COPE; STXBP3; TUSC2; CBX3; EXOC3; GABARAP; RNF13; TWF2; GABARAPL2; STAT1; NUPL2; ZNF236; OGFR; ATF6; PAXIP1; CASC3; RALY; BRD3; DDX42; TARDBP; COMMD3; CCT5; DGAT1; ELL2; PGLS; ABCB10; MACF1; ADAT1; PRDXS; AP3M1; APPL1; CD3EAP; DNPEP; ARL2BP; AHSA1; CCRN4L; CD2AP; COPG2; FAM50B; AATF; SERGEF; CCNDBP1; FBXL3; FBXL4; FBXL6; FBXW2; FBXO22; FBXW8; FBXO3; FBXO8; FKBP8; TIMM10B; EIF2C1; GRHPR; GTF3C4; HNRNPH3; HARS2; MID2; NUBP2; MSRB2; POMZP3; PRDM2; RYBP; SCAP; SNW1; XRN2; ZNF212; HACL1; RHBDD3; ZNF346; FTSJ1; KEAP1; G3BP2; FBXW11; KIN; KPNA6; LETM1; PLA2G15; PIGN; DNAJB9; GTPBP4; NUFIP1; FBXO9; TTC33; BLOC1S6; PEF1; PFAS; PFDN2; CDK14; PITPNB; ANP32C; ICMT; PRDM4; ZMYND8; H2AFV; RAB3GAP2; RLF; RSU1; SF3B3; SEC22A; SNAPIN; STATSB; TIMM10; TIMM13; TIMM8B; TIMM9; ATP6V0A2; PRPF6; TXN2; UCK2; WBP1; WBP2; YWHAG; ZNF281; EIF3K; DNAJC15; N6AMT1; C16orf80; VPS4A; HTRA2; NXT1; TBK1; SAP30BP; VPS51; MAT2B; POLM; GNL2; RBM15B; CPSF1; TRA2A; SAC3D1; CCDC106; EEF2K; SNX15; PRRC2B; UBIAD1; SNX8; SNX11; ATG4B; PAXBP1; NME7; GMPPB; GMPPA; SEC61A1; TIMM22; ALG6; TFPT; KCNJ14; NENF; CNOT7; ZNF225; ANAPC2; ANAPC4; ABT1; DPP7; PREB; NRBP1; FTSJ2; USP25; UBQLN1; STOML2; ST6GALNAC6; UBQLN2; BAZ1A; BAZ2A; BAZ2B; DHX38; CCDC22; SNRNP200; DEXI; SACM1L; MRPS28; WDR37; DCPS; OSTM1; ASF1A; SNX24; SPCS1; ANAPC15; UNC50; MRPS18B; C19orf53; MKL2; ACAD9; MRPL42; NOB1; NTMT1; ASTE1; FAM32A; MRPL13; ZNF770; C16orf72; ZC3H7A; ZBTB44; SETD2; MRPL18; NDUFAF4; CCDC59; METTLS; CHMP4A; GTPBP8; CRIPT; MRPL15; TIMM21; LGALSL; ORMDL2; DYNLRB1; CNIH4; TMEM208; SSU72; AP2A1; TMEM258; NDUFA8; PPP2R1A; VAMP2; HSD17B8; UBL4A; GNPAT; EIF2B2; RAPGEF2; RBX1; TMEM5; CNPY2; Cllorf58; MGAT4B; DNAJC8; SUCO; EXOSC2; NOMO1; TRAM1; CAPN7; ETHEl; BRD4; ISCU; TGDS; C22orf28; TMEM50A; KLHDC2; PDSS1; PATZ1; EDC4; PPIL2; PISD; MTCH2; ZNF318; TBC1D22A; ZNF324; HIBCH; GNL3; FAM162A; AKAP8L; RNF11; ACAD8; DIEXF; PELP1; SND1; GHITM; VPS41; UQCRQ; ZBTB11; AFF4; INVS; SNX5; TUBGCP4; CHMP2A; RNF115; KLHL20; LSM1; LSM3; DIMT1; ZNF330; TNRC6A; GOLIM4; PRPF19; UTP20; RABGEF1; TOR1B; MCAT; CNOT3; ZNF232; TMOD3; ZKSCAN5; LATS2; BRD1; ERO1L; ZNRD1; DNTTIP2; MAGED2; PIK3R4; UBXN4; MDN1; FAM120A; FAF2; PSME4; ATP11B; ZNF592; SH3PXD2A; CTR9; TTC37; MDC1; SAFB2; SLC25A44; TTI1; PHF14; KDM4A; UBE3C; EMC2; KIAA0100; KIAA0355; AQR; TMEM63A; CEP104; SART3; USP34; SETD1A; LAPTM4A; SLK; MLL4; MLEC; KIAA0195; EIF4A3; TM9SF4; MTSS1; SPCS2; BMS1; PTDSS1; SERTAD2; MAML1; SNX19; TATDN2; MRPL19; TOMM20; EFCAB14; URB2; TSC22D2; ARHGEF11; ZBTB24; PLEKHM1; C2CD5; ZNF518A; EPM2AIP1; C2CD2L; FARP2; CEP350; LRIG2; PJA2; TOMM70A; SEC24D; FCHSD2; URB1; ZC3H11A; TOX4; DDX46; ZBTB39; OSBPL2; ZBED4; FIG. 4; KIAA0196; AP5Z1; DENND4B; SUPT7L; FAM20B; RNF10; ZBTB5; JOSD1; HELZ; KIAA0020; N4BP2L2; PDAP1; SCAF8; ZFP30; DOLK; AAK1; LMTK2; ICK; R3HDM2; ZNF510; PPP6R1; MLXIP; TRAPPC8; MON1B; MORC2; ZHX2; KIAA0907; BAHD1; DHX30; TCF25; PDCD11; PCNX; HMGXB3; RALGAPA1; WDFY3; RAB21; SPEN; FBX021; EXOSC7; KDM4B; USP33; PHLPP2; ZNF292; XPO7; MON2; PDXDC1; FRYL; PDS5B; ZHX3; KIAA0754; PIKFYVE; ZNF609; TBC1D9B; GGA2; WAPAL; SETX; SETD1B; FTSJD2; ERP44; RRP1B; MYCBP2; AVL9; PPRC1; ZC3H13; SARM1; CDK12; MRPS27; CUL9; FAM179B; SMG1; TAB2; PLXND1; ATG2A; RAD54L2; SMC5; MAST2; ZZEF1; ANKLE2; ZC3H3; GRAMD4; CIC; TBC1D9; WDR43; SNX13; MPRIP; NUP205; EFR3A; RTF1; TTLL12; METAP1; ZCCHC14; CEP68; PHF3; LARP4B; RCOR1; FAM168A; PMPCA; PLEKHM2; ZC3H4; RRS1; PRRC2C; TBC1D12; DNAJC9; KIAA0556; RPRD2; ATP11A; DNMBP; POFUT2; CLUH; NUP160; CSTF2T; ATMIN; KIF13B; FKBP15; SIN3B; NCAPD3; DNAJC13; MAN2B2; KIAA1033; USP22; DPY19L1; SZT2; WDR7; VPS39; DNAJC16; KHNYN; ANGEL1; USP24; FNBP4; KIAA1109; LARP1; PPP1R13B; PUM2; UFL1; RRP8; KIAA0947; SMG5; MAU2; NCSTN; NUDCD3; MED13L; ZDHHC17; ADNP; LARS2; PPWD1; ZFYVE26; TMEM131; GLTSCR1L; POFUT1; SUZ12; SCRIB; MORC3; SKIV2L2; R3HDM1; ELP5; PANX1; VPS13D; SAMM50; HECTD1; NIPBL; YIPF3; TECPR1; DCAF12; ABHD14A; EP400; C3orf17; DCAF13; TMEM186; AASDHPPT; POLR1A; CCDC28A; AHCTF1; CAMSAP1; CNOT6; NELFB; ZDHHC5; MTMR9; ATL3; NOL11; PTPN23; NIPSNAP3A; HEATR5A; FAM98A; SLC22A23; KBTBD2; SYF2; PNISR; KIAA1429; NECAP1; DHRS7B; IBTK; TBC1D10B; RNF167; C2CD3; DAK; ZZZ3; RPAP1; LRIG1; UPF2; PTCD1; GLCE; OPAl; UBXN7; LTN1; POLDIP2; GPATCH4; HERC4; CCDC9; CCZ1; LDLRAP1; PRPF31; EPC2; GAPVD1; TRPC4AP; IRF2BP1; C10orf12; NAT9; ZNF337; NOC2L; RSL1D1; GTPBP5; SENP3; TRUB2; WWC3; ZNF777; BRPF3; COQ2; GPKOW; MMADHC; RRP7A; DESI1; SGSM3; GLTSCR1; DCAF8; WARS2; UBXN1; GTF2A1; ZNF593; AZIN1; MBTPS2; PCF11; CDC40; ZBTB7A; UBR5; EIF5B; TRIM33; LAP3; NBAS; WDPCP; TXNDC12; TXNDC11; POP5; RPS27L; POMP; TMA7; NOP58; NMD3; TRMT6; ATP6V1H; MTERFD1; SLC35C2; PELO; GET4; MRPL2; DERA; MRPL4; APIP; CUTC; FCF1; NDUFA13; ERGIC3; MRPS17; MRPS7; TAF9B; UBE2D4; HEBP1; ATP6V1D; ADIPOR1; UTP18; ABHD5; NDUFAF1; PHF20L1; TFB1M; UBE2J1; RBMX2; LACTB2; SUV420H1; TRAPPC12; RMDN1; MRPS2; COQ4; UTP11L; SBDS; C14orf166; DERL2; FAHD2A; EXOSC1; SF3B14; ISOC1; EMC9; MRPL11; MRPL48; TMBIM4; TPRKB; PPIL1; MED31; FAM96B; MRPS16; MRPS18C; FIS1; PAM16; MRPS23; MRPS33; GOLT1B; BOLA1; VPS36; PTRH2; TVP23B; GLOD4; CDK5RAP1; STYXL1; RBM7; RPL26L1; COMMD2; IER3IP1; NAA20; ZFR; TELO2; RLIM; TMEM66; COPG1; RAB10; INSIG2; CHCHD2; DYNC1LI1; HSD17B12; COMMD10; WDR83OS; TRAPPC4; RAB4B; PIAS1; NOL7; HEMK1; SDF4; MRTO4; LSM7; NAA38; PDGFC; CPSF3; VPS28; TRAPPC2L; TRIP4; DBR1; POLK; MAN1B1; DDX41; SNX9; VPS29; NLK; BIRC6; FAM8A1; NAGPA; TUBE1; SELT; TAOK3; HP1BP3; PCYOX1; HSPA14; RSL24D1; SS18L2; DNAJB11; POLR3K; ATPIF1; WBP11; RAB14; ZNF274; ZNF639; SRRM2; ZDHHC2; DDX47; TACO1; ACP6; WWOX; AKAP7; C9orf114; CTDSPL2; TRIAP1; C11orf73; CWC15; TRMT112; UFC1; RTFDC1; GLRX5; RNF141; GLTP; RTEL1; NCKIPSD; EMC4; TMEM9; CXXC5; ANKRD39; C20orf111; CCDC174; ZC3HC1; C9orf156; PDZD11; VTA1; TMEM69; MRPL37; RNF181; MRPL51; PBDC1; MRPL27; ZCCHC17; KBTBD4; SCLY; C9orf78; KLF3; TM7SF3; SCAND1; BFAR; COA4; BCCIP; ERGIC2; RSF1; TIMMDC1; KDM3B; ARMCX3; TDP2; KRCC1; ZNF644; MRPL35; WAC; MRPS30; GDE1; CRNKL1; STX18; POLA1; RWDD2B; SEPSECS; USP18; NUP54; PTOV1; CPSF2; POLE3; CHRAC1; MRPL39; TMED9; HAUS7; ARID1B; MPHOSPH8; POGK; CNOT11; FOXRED1; MIER2; INO80; ZRANB1; UBE2Q1; TRIM44; WDR5; ZC3H7B; MED29; BMP2K; VEZT; ZCCHC8; RNPC3; ALKBH4; C17orf59; CNNM3; CDKN2AIP; KCTD9; KLHL24; TRIT1; FTSJ3; CNNM2; DYM; KLHL28; GATAD2A; ANKRD10; ZCCHC10; OTUB1; TRPM7; GIN1; MCM9; FBXL12; ANKRD49; WDR55; PGPEP1; TASP1; ZNF3; CC2D1A; TMEM104; QRICH1; THUMPD1; ZCCHC2; DPP8; ST7L; CWC25; UHRF1BP1; ALKBH5; PNRC2; MTMR10; SLC39A4; LRRC40; PXK; TBC1D22B; CDKAL1; CHD7; FAM208B; FOCAD; BTBD2; YTHDF1; HEATR2; OSGEP; ZSCAN32; UBE2R2; CHCHD3; IMPAD1; RAB20; WRAP73; TRMT10C; EXD3; KANSL2; MARCH5; ADPRHL2; COMMD4; CECR5; FAM206A; MRPL16; SDHAF2; SLC48A1; TRNAU1AP; FAM120C; Clorf109; PARP16; SSH3; INTS8; C4orf27; THG1L; SLC25A38; SLC35F6; ZNF416; CLN6; PINX1; Clorf123; VPS13B; PRPF40A; DDX27; GIDS; HIF IAN; TMCO3; PAK1IP1; LAMTOR1; ZNF446; TRMT61B; CDC37L1; C19orf24; PIH1D1; PPP2R3C; STX17; NPLOC4; PRPF39; C14orf119; DENND4C; GPATCH2L; PHIP; USP47; PTCD3; TRMT12; VPS37C; IWS1; NRDE2; MRPL20; RUFY2; SCYL2; TMEM248; RNF31; TRMU; ARGLU1; ClOorf118; MED9; YEATS2; WDYHV1; GPATCH1; SAMD4B; WDR6; LUC7L; WDR70; ATG2B; GPATCH2; SLFN12; AGGF1; RBM22; MAGOHB; PLEKHJ1; MANSC1; WDR60; VAC14; TMEM39B; IARS2; PRPF38B; AKIRIN2; GPN2; ARHGEF40; HEATR1; TRIM68; CCDC94; LARP1B; SRBD1; IPO9; ELP3; WDR74; GSPT2; NLE1; THAP1; MTPAP; LMBR1L; SDAD1; WDR11; ARMC1; DARS2; TMEM33; TSR1; PNPO; SHQ1; MRPS10; INTS10; RMDN3; RNMTL1; SMG8; RNF220; RIC8B; SLC4A1AP; NADSYN1; DNAJC17; ASUN; RPRD1A; MAP1S; N4BP2; GOLPH3L; ATF7IP; DHX32; ARL8B; ZFP64; DNAJC11; HMG20A; TBC1D13; TMEM57; VPS35; ARFGAP1; PANK4; USP40; COA1; SMU1; UBA6; AP5M1; NUP133; SLC38A7; OGFOD1; CCAR1; AGK; TMEM184C; CCDC25; WDR12; TTC17; TYW1; TMEM39A; WDR41; ADI1; THNSL2; TMEM19; NUDT15; IMP3; PHF10; QRSL1; ZNF654; CWF19L1; EXOC2; BRF2; PBRM1; CCDC91; RNF121; BRIX1; DDX19A; RFK; C6orf70; RSAD1; FGD6; TMA16; C5orf22; ABCF3; UFSP2; LIN7C; RSBN1; BLOC1S4; LMBRD1; SYNJ2BP; LSG1; METTL2B; DCP1A; COPRS; ST7; PI4K2A; TMEM63B; RRN3; UTP6; BDP1; RNF130; FBXO6; IMPACT; VIMP; EMC3; CAND1; UBAP2; TMEM242; EAPP; PPP2R2D; BRK1; ITFG2; CISD1; PLGRKT; USE1; TEX2; ZC3H15; TMEM165; ACTR10; ASH1L; TMCO6; LRRC59; KIAA1704; CSGALNACT2; WSB2; NOP10; SLC35E3; ZNF395; VPS33B; RNF114; CMAS; BIN3; FAM114A2; DHTKD1; COG1; MAML3; TRPV1; SLC25A40; MKKS; PCDHGB5; CLN8; NANS; UBB; DAZAP1; BRWD1; TERF2IP; SLC38A2; YIPF1; GAR1; SSH1; RBM27; KCTD5; FBXO42; MRPS21; FBXW5; ETAA1; ANKIB1; MIOS; SMCR7L; TOLLIP; TMX3; HEATR5B; DHX29; EXOSC4; ELP4; PUS7; CCDC93; ASNSD1; MRPL50; FAM35A; TOMM7; WDR5B; DDX49; ING3; TRMT13; VSIG10; GTPBP2; LIN37; C19orf10; SMG9; ALG1; UBFD1; TMEM234; PPP1R37; MOSPD1; YLPM1; RNF20; GPCPD1; FAM214A; WDR45B; METTL3; GSK3A; CHST7; DIABLO; INPP5E; POLE4; LARS; UGGT1; UGGT2; KCMF1; TM9SF3; UBQLN4; WRNIP1; GRIPAP1; BDH2; TMEM167B; PNO1; SH3GLB2; STARD7; EMC7; C1GALT1; EXOSC5; MCCC1; NCLN; FEM1C; DUSP22; CMC2; MRPS22; YAE1D1; C11orf30; MFF; SDR39U1; XAB2; CCDC47; C5orf15; NIT2; OTUD7B; PARP6; RNPEP; FAM20C; PRDM10; PPAN; PSMG2; ADPRM; MRPL1; TOMM22; CHPT1; CCNL1; MNT; CIAPIN1; C16orf62; ANKMY2; RARS2; RALGAPB; ZMIZ1; RALGAPA2; NKIRAS1; ENTPD7; PCNP; PITHD1; PARP11; UTP3; AVEN; C12orf4; C12orf5; MAN1C1; PDSS2; SETD8; REXO4; NUP107; MRPL47; ATP13A1; DDX24; SCYL3; SEPN1; ATP10D; TUBGCP6; LYRM2; SNX14; YIF1A; GALNT1; MCOLN1; CSRP2BP; TMEM9B; MRS2; CLK4; RAB22A; ANKHD1-EIF4EBP3; REXO1; KIAA1143; GATAD2B; LRRC47; ZNF512B; ZNF490; USP31; PRR12; ATXN7L1; NLN; ESYT2; KIDINS220; MTA3; AARS2; INTS2; XPO5; ARHGAP31; SERINC1; UBR4; NUFIP2; MIB1; ZNF398; KLHL42; PDP2; USP35; KLHL8; TMEM181; ARHGAP21; CRAMP1L; KIAA1430; WDFY1; ZNF687; WDR48; FNIP2; PITPNM2; SLAIN2; RANBP10; KIAA1468; VPS18; ZBTB2; SH3RF1; PHRF1; RDH14; FLYWCH1; ALS2; ZSWIM6; KIAA1586; DDX55; CWC22; GBA2; DENND1A; KIAA1609; ANO8; METTL14; EPG5; NCOA5; PPM1A; DHRS4; DEAF1; UBC; RAP2A; ZNFX1; MBNL1; ZNF253; NDUFV2; KAT2A; NMT1; ZNF8; MTMR3; MRPS12; POLR2L; PPA1; PPIA; MRPL23; TNFAIP1; TRAF2; KDM6A; XRCC5; ZNF273; TMX4; GATAD1; KIAA1967; LSM2; CCNB1IP1; C6orf47; SLC30A1; SRPRB; ENOPH1; RPRD1B; ZNF77; PRUNE; SCAF1; SELK; RBM25; WIZ; RRAGD; SNX6; TRIM39; C21orf59; ZFYVE1; SENP2; PDLIM2; KLHL12; GPBP1L1; C12orf10; UTP14C; ZNF500; VPS11; SAV1; CCDC90B; FASTKD5; GUF1; SPCS3; RINT1; RIC8A; MIIP; EEFSEC; TRAPPC11; ZFAND3; SRR; PPP1R11; ZNF148; POLR2F; ZNF277; ITM2B; TIA1; FBXW4; ABHD4; MRPL17; UBE2O; HEATR6; NSUN3; CERS2; GPATCH3; HPS4; GALNT11; ZNF335; MRPS14; PCIF1; FKBPL; RBM26; GOLPH3; MCCC2; SNX16; MAGEF1; TMBIM1; DUS1L; MRPL46; XYLT2; EIF4H; Cllorf24; ZFYVE20; PDF; C17orf75; OSGEPL1; MMS19; DNAJC1; TFB2M; TOR3A; HERPUD2; NOC3L; RNF25; NSD1; LMBR1; XPO4; HS1BP3; IKZF4; ZMAT3; KLHL25; GZF1; C5orf28; TMEM168; ATG3; POLR1E; SUDS3; TTC31; NARFL; ZDHHC6; PCNXL4; ACTR6; MRPS25; DNMT3A; VPS52; GIGYF1; VPS16; ANAPC1; SNRNP35; DGCR14; COPS7B; NUCKS1; ACBD3; TNS3; FAM160B2; PARP12; ZNF574; SFXN1; IPPK; CCDC14; C6orf106; C11orf1; RMND5B; CERK; LMF1; OSBPL11; RMND5A; MPHOSPH9; ARV1; NMNAT1; MAP1LC3B; PORCN; MARCH7; YTHDC2; TUT1; MRPS11; RFX7; PAPOLG; C12orf43; ACTR8; CASD1; CCDC71; MRPL44; VPS33A; NOL6; KRI1; UPF3B; UPF3A; RSRC2; INTS3; FRY; ANKRA2; SPATS2; ZNF649; SELRC1; UBE2Z; C8orf33; CAPN10; ZNF747; FUNDC2; DDRGKl; MRPS34; MRPL34; CDK11A; MRP63; YIPF2; PRR14; C19orf43; CUEDC2; METRN; DDX50; DDA1; NUP37; SPATA5L1; PDCL3; ERI3; C7orf26; NABP2; SECISBP2; NOC4L; METTL16; FASTKD3; TMEM109; C2orf49; ASB8; DCTPP1; Clorf50; CCDC86; C11orf48; WDR18; WDR77; SLC25A23; SMIM7; ALG12; C9orf16; TAF1D; DHX58; TMEM185B; FAM134A; PHF23; PPDPF; DHRS11; GNPTAB; NOL12; LENG1; Clorf35; RBM42; ZNF343; FBXL15; DCAF10; NDUFS7; PGS1; IRF2BPL; LRFN3; HAUS3; CYP2R1; PAGR1; C2orf47; GCC1; ATP13A3; ABHD8; NKAP; CDC73; CARS2; MRPL24; C10orf76; MULl; RNF219; ADIPOR2; FAM118B; TANGO6; SNRNP25; C6orf211; OCEL1; ARMC7; OSBPL9; ROGDI; CHMP6; SRD5A3; PANK3; HECTD3; NLRX1; FN3KRP; C22orf29; ZDHHC14; MSANTD2; NAA35; YRDC; MANEA; OGFOD3; BBS1; PRKRIP1; NOL9; TBL1XR1; ZNF768; THAP9; PALB2; TEFM; AAMDC; BBS10; SNIP1; ASB13; ASB7; KATNBL1; TXNDC15; CCDC82; KLHL36; FBX031; HPS6; TTC21B; PTCD2; CAMKMT; METTLE; ZMYM1; GEMIN6; NHEJ1; ZBTB3; TMEM180; CSPP1; RPAP2; CBLL1; RABEP2; UBA5; TGS1; GGNBP2; ZNF672; NUP85; EIF2C3; PYROXD1; ACTR5; MRM1; KIAA0319L; SLC35E1; OBFC1; ZCCHC4; C10orf88; RMI1; FAM192A; PHC3; WWC2; NAA25; UBTD1; TMEM62; PANK2; FBXL18; GFM1; KLHL18; ZNF606; MZT2B; VCPIP1; RPF1; THOC7; CENPT; USP36; CTC1; MUS81; WDR19; CHD9; PROSER1; CCDC92; TM2D3; NAA50; COQ10B; ACSF2; C17orf70; SIK3; SLC35F5; FAM214B; C16orf70; EDEM3; ITPKC; GRPEL1; MED28; DNAJC5; WDR82; WDR61; TNKS2; THUMPD2; NDFIP1; CYB5B; ZNF34; WDR59; KLHL15; INTS5; EEPD1; DUSP16; SH3BP5L; SETD7; ACAP3; KIAA1715; MAP2K2; RAIl; TMX1; ILKAP; SLC25A32; CLPTM1L; PTDSS2; HM13; ITFG1; SGPP1; WBSCR16; Clorf21; CSRNP2; MRPS26; ANKRD13C; CCDC130; PLA2G12A; CTNNBL1; APOL2; TRIMS; SNX27; C6orf62; ISCA1; TRIM56; SBF2; MED25; SHARPIN; ARPC5L; RAB1B; QTRT1; SLC25A28; HDHD3; NECAB3; MRPS15; SF3B5; INO80B; RAB33B; HUWE1; MRPL9; RILP; COG3; GUCD1; ZMIZ2; FAM103A1; SELO; RIOK1; GRWD1; L3MBTL2; LONP2; RBM4B; BBS2; GORASP1; MRPS5; MRPL32; FRMD8; ATAD3B; TAF3; RSPH3; TMEM120A; SNX25; MRPS24; RNF26; STK40; ClOorf11; EIF2A; TM2D1; ITFG3; SRSF8; MRPL14; MRPL43; RBM48; MAGT1; HDHD2; TMEM222; SLC10A7; KBTBD7; ANKRD27; ENKD1; CEP192; PCBD2; ZNF394; ATRIP; WDR75; USP42; TOMM40L; UTP15; PHAX; SLC7A6OS; FAM175B; KATE; RNASEH2C; RPF2; SON; ANKRD17; CHD6; PCNXL3; ZCCHC7; SETD3; SGK196; TMEM117; WDR24; ZNRF1; TRAF7; MAF1; MED10; SLC37A3; DCUN1D5; POLR3GL; C9orf64; CHCHD5; C9orf89; POLDIP3; YIPF4; NOA1; COQ5; NICN1; PRADC1; BTBD10; TMEM79; NTPCR; TMEM175; ZDHHC16; ING5; UTP23; LLPH; MIEN1; MNF1; PDCD2L; MRPL45; BRMS1L; VPS25; LSMD1; ACBD6; DNAJC14; LZIC; APOPT1; TMEM101; ELOF1; GFM2; COG5; HPS3; C5orf4; MKI67IP; BAZ1B; PINK1; HOOK3; MSANTD4; SYVN1; ZNF333; FAM120B; CC2D1B; ZNF527; PPIL3; MRPS6; MRPL41; MRPL38; MRPL36; C14orf142; JAGN1; ZC3H8; MAK16; GNPTG; USP38; HIATL1; SMEK1; GLYR1; DPY30; FAM126A; USP32; HINT2; MCEE; LOXL3; USP30; FUT10; PCGF1; MPV17L2; TUBA1C; MFSD9; TXNDC17; LMNB2; PHF5A; LRCH3; KLHL22; CCDC142; CBR4; ZC3H10; PARP10; ZBTB45; SYAP1; SPPL2A; ADO; GTDC2; FAM73B; ATAD1; TBRG1; NFATC2IP; CEP89; ZNF341; FAM136A; TMEM87B; CIRH1A; PPP1R15B; FIZ1; DIRC2; SPRYD3; TMEM209; C8orf76; C12orf52; ATG4C; MUM1; WDR73; LACTB; ABHD13; LTV1; SERAC1; TIGD5; PRPF38A; ALKBH6; LSM10; ATG4D; PPP1R16A; PYURF; UBL7; TMEM128; TMEM141; TMEM60; C9orf37; POLR2C; CSRNP1; HIAT1; SYNE1; SARNP; EAF1; ALG2; ZCCHC3; PNPT1; RRP36; ZCRB1; NEK9; RBM18; SURF4; PIGS; LMF2; PPP1R3F; PURB; DGCR6L; BTBD6; MRPS36; C22orf32; MICALL1; KIAA1731; ZNF622; IMP4; METTL18; PGAP3; C9orf123; CDK11B; TPGS1; MFN1; INTS4; TRIM41; TP53RK; N4BP2L1; MMAB; CCDC97; GADD45GIP1; ADCK2; ZNF830; RFT1; MGME1; VPS26B; NACC1; MBD6; ESCO1; SMYD4; ATG4A; WDFY2; DNTTIP1; RBM33; TMEM203; EGLN2; MRPL53; SNAP47; TADA1; THEM4; GLMN; ANKH; KLHDC3; NAA15; TSR2; UBE2J2; LOH12CR1; SMIM11; FAM207A; RPUSD1; ZNF354B; MY018A; SLC36A1; SCAMP4; PIGU; SLC44A1; ZSWIM1; B3GALT6; MED30; TMEM41A; CDKN2AIPNL; SLC35A4; DYNLL2; UBE2F; SRXN1; B3GAT2; ROMO1; DTD1; FAM210B; OVCA2; SPSB3; SOCS4; PRRC1; ELMO2; LRPPRC; WIPF2; RSPRY1; ZNF526; ZNF721; SAT2; HELQ; MED22; RAD52; NUP35; SPTSSA; PYGO2; FAM122A; KLC4; KIAA2013; FAM105B; SAMD1; C19orf52; CEP95; PRMT10; TTC5; OXNAD1; MTG1; G6PC3; TMEM183A; MARS2; NOM1; MVB12A; GTF3C6; KTI12; FAM195A; SAAL1; CASC4; C12orf57; MFSD3; MALSU1; ACYP2; BATF2; NUS1; GLI4; CDAN1; CYHR1; TECR; HINT3; TAF8; HAS3; PPP1R14B; MPLKIP; NDNL2; RHOT2; SLC25A46; ALKBH8; WDR85; ZNF653; GINM1; LEO1; ANKRD54; MITD1; TAMM41; HIGD2A; MSI2; SPPL3; PPIL4; ALKBH3; FGD4; MTFMT; PPM1L; TSTD2; EHD4; ORMDL3; WDR36; PPTC7; RPIA; SLC39A3; ANGEL2; HN1L; MAPK1IP1L; L3HYPDH; TEX261; LRRC28; FOPNL; ZC3H18; FLCN; CYB5D1; TBC1D20; TMEM42; NACC2; FAM76B; ZNF18; ZNF480; ZNF420; ZNF558; ZNF570; BROX; LSM14B; PUS10; SEPT10; CCDC12; SPICE1; THAP6; ZMAT2; APOA1BP; MBNL2; FAM91A1; DENND5B; ZNF564; IMMP1L; ZFC3H1; LRRC45; TSNARE1; CCNY; UBLCP1; UPRT; FUK; ZUFSP; OARD1; NSMCE1; FAM200A; ZSCAN25; SFT2D1; MAP2K7; NAPRT1; CSNK1A1L; VTI1A; MRPL30; OMA1; FRA10AC1; UBALD1; MRPL10; CCDC127; NUDCD2; C6orf57; ZBTB49; SLC15A4; ATPAF2; KIFC2; ABTB2; ZNF511; MTPN; CRYZL1; ZNF23; ZSCAN21; ZNRF2; SGMS1; RPP25L; SVIP; RPUSD2; C12orf23; CHMP7; ZNF585B; ARRDC1; ORAI3; ZNF561; TADA2B; TRMT61A; SLC36A4; ARL14EP; C12orf45; TARSL2; SPATA2L; LSM12; ZNF491; ZNF440; Clorf131; KCTD18; METTL6; GRPEL2; ZNF786; NDUFAF6; TMEM68; HGSNAT; ARHGAP42; KBTBD3; CWF19L2; C12orf66; LYSMD4; ZSCAN29; ZNF785; TMEM199; ZNF417; C19orf25; B3GALNT2; ZNF362; MROH8; COMMD1; KANSL1L; XXYLT1; SCFD2; TRMT44; SRFBP1; SNRNP48; ZNF579; ZNF383; SDE2; RNF168; MIER3; TCEANC; ARID2; UBE2E2; NANP; DENND6A; RWDD4; CCDC111; HIPK1; SENP5; STT3A; PATL1; EFHA1; CPNE2; NT5DC1; C6orf89; HIBADH; BRAT1; RICTOR; YTHDF3; TMEM256; MFSD8; D2HGDH; TAB3; TMEM18; UHRF2; TANGO2; N4BP1; TCEANC2; EID2; NPHP3; ZNF461; LRRC57; CNEP1R1; PUSL1; TMEM161B; ZNF791; TAPT1; KIAA1919; LNX2; AGXT2L2; MED19; COG7; CRYBG3; CPNE8; PIGP; ZFP1; C2orf69; ZNF367; AAED1; KDELC2; TTL; CACUL1; ZFPM1; MLL3; MLX; Cllorf31; PGBD3; TRIM35; HSCB; CBWD2; RC3H1; TNFSF12-TNFSF13; SUGP1; MMAA; MRPL54; PSENEN; RUNDC1; FAM149B1; MMGT1; DCUN1D3; CCDC117; ZNF584; KCTD20; PRR14L; ANKRD52; DIP2B; INO80E; HEXDC; RTTN; ZNF776; SLC9A9; C3orf33; DCBLD1; NSMCE2; PDZD8; BLOC1S2; TTC9C; FAM126B; C3orf38; RABL3; COX18; SREK1IP1; KRTCAP2; NDUFAF2; PPP4R2; CCDC50; TMEM167A; NOP9; UBR1; ADCK5; N6AMT2; GPATCH11; ZNF575; EMC10; DDX51; UBR7; TXLNA; EXOC8; ZADH2; CRIPAK; C5orf51; CDK5RAP3; CHMP4B; ZNF800; GATC; INADL; NR2C2AP; MIDN; NUDT14; CYP20A1; P4HTM; PDE12; PPM1G; TUBB; GGT7; ERC1; FAM134C; SLC35B2; ZNF598; MRPL52; GMCL1; DRAM2; PIGW; ZNF616; ZBTB8OS; ZNF678; ZDHHC21; MTDH; ARL5B; AGPAT6; STT3B; GPR180; ZACN; MRPL55; GCC2; ZNF445; EXOSC8; MRPL21; AUP1; C17orf58; OGT; QSOX2; LYRM7; DNAJC24; BCDIN3D; GRASP; UBXN2A; CRTC2; METTL2A; TMTC3; DPY19L4; AASDH; TMED7; ZSCAN22; ZSCAN2; COQ6; USP12; ZNF227; ZNF428; MTERFD2; C9orf85; CMC1; ZNF595; NSUN6; TMED4; BRICD5; PDDC1; C15orf38; MRPS9; TPRG1L; TRNT1; TICAM1; HEATR3; ZNF326; CYP2U1; C9orf142; ARRDC4; HNRNPA3; DND1; ISCA2; SPTY2D1; RPS19BP1; PHLPP1; RNF126; C7orf55; TSC22D3; GNPNAT1; COX20; Clorf52; CCZ1B; GANC; ARSK; E2F6; LYSMD3; GANAB; APOOL; RSBN1L; C19orf54; RPL7L1; CCDC84; FAM174A; NHLRC2; ZNF710; HDDC3; ATP9B; ZNF773; MIA3; TMEM110; ACACA; FAM120AOS; NUP43; SS18L1; DHX57; NELFCD; NSUN4; NDUFAF3; CARM1; TMEM189-UBE2V1; CCDC137; NACA2; PHF17; FAHD2B; TMEM179B; CCDC23; FAM86A; SLC25A35; RP9; POLR1C; CHCHD1; RAPH1; TMEM81; RBM12B; MBLAC1; MRFAP1L1; COMMD6; C19orf70; CLYBL; MRAP; RNF216; GTF2H5; FAM199X; ERICH1; ZDHHC24; TSEN54; CYP4V2; C1orf174; BLOC1S3; METTL10; ZNF543; ZNF789; ZNF517; SFXN4; and any combinations thereof. In some embodiments, the reference gene(s) is/are analyzed by additional qPCR.

In some embodiments, the in-process control is an in-process control for reverse transcriptase and/or PCR performance. These in-process controls include, by way of non-limiting examples, a reference RNA (also referred to herein as ref. RNA), that is spiked in after RNA isolation and prior to reverse transcription. In some embodiments, the ref. RNA is a control such as Qbeta. In some embodiments, the ref RNA is analyzed by additional PCR.

In some embodiments, the extracted nucleic acids, e.g., exoRNA, are further analyzed based on detection of an ALK fusion transcript, e.g., an EML-ALK fusion transcript.

In some embodiments, the further analysis is performed using machine-learning based modeling, data mining methods, and/or statistical analysis. In some embodiments, the data is analyzed to identify or predict disease outcome of the patient. In some embodiments, the data is analyzed to stratify the patient within a patient population. In some embodiments, the data is analyzed to identify or predict whether the patient is resistant to treatment. In some embodiments, the data is used to measure progression-free survival progress of the subject.

In some embodiments, the data is analyzed to select a treatment option for the subject when the ALK fusion transcript, e.g., an EML-ALK fusion transcript, is detected. In some embodiments, the treatment option is treatment with crizotinib (Xalkori). In some embodiments, the treatment option is treatment with ceritinib (Zykadia) or alectinib (Alecensa) if crizotinib stops working or is not well tolerated. In some embodiments, the treatment option is treatment with a combination of therapies.

Various aspects and embodiments of the invention will now be described in detail. It will be appreciated that modification of the details may be made without departing from the scope of the invention. Further, unless otherwise required by context, singular terms shall include pluralities and plural terms shall include the singular.

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representations as to the contents of these documents are based on the information available to the applicants and do not constitute any admission as to the correctness of the dates or contents of these documents.

### BRIEF DESCRIPTION OF THE FIGURES

FIG. 1 is a graph that depicts the distribution of EML4-ALK variants in non-small cell lung cancer (NSCLC). This figure has been adapted from Ou et al., Crizotinib for the treatment of ALK-rearranged non-small cell lung cancer: a success story to usher in the second decade of molecular targeted therapy in oncology, The Oncologist, vol. 17(11): 1351-75 (2012).

FIG. 2 is a schematic representation of the EXO501a workflow for detection of EML4-ALK fusion transcripts from plasma.

FIG. 3 is a graph depicting EXO501a analysis of tissue-correlated NSCLC plasma samples.

FIGS. 4A, 4B, and 4C are a series of graphs depicting EXO501a standard curves for detection of each EML4-ALK variant (FIG. 4A: v1; FIG. 4B: v2; and FIG. 4C: v3a,b,c).

FIG. 5 is a graph depicting the comparison of EXO501a assay with two alternative tests for detection of cell line-derived EML4-ALK v1 fusion transcript.

### DETAILED DESCRIPTION OF THE INVENTION

The present disclosure provides methods of detecting one or more biomarkers, such as an ALK fusion transcript, in a biological sample to aid in diagnosis, prognosis, monitoring, or therapy selection for a disease such as, for example, cancer. In some embodiments, the cancer is a lung cancer. In some embodiments, the cancer is non-small cell lung cancer (NSCLC).

The methods and kits provided herein are useful in detecting an EML-ALK fusion transcript in plasma samples. In some embodiments, the ALK fusion transcript is an EML4-ALK fusion transcript. In some embodiments, the EML4-ALK fusion transcript is EML4-ALK v1, EML4-ALK v2, EML4-ALK v3, and any combination thereof.

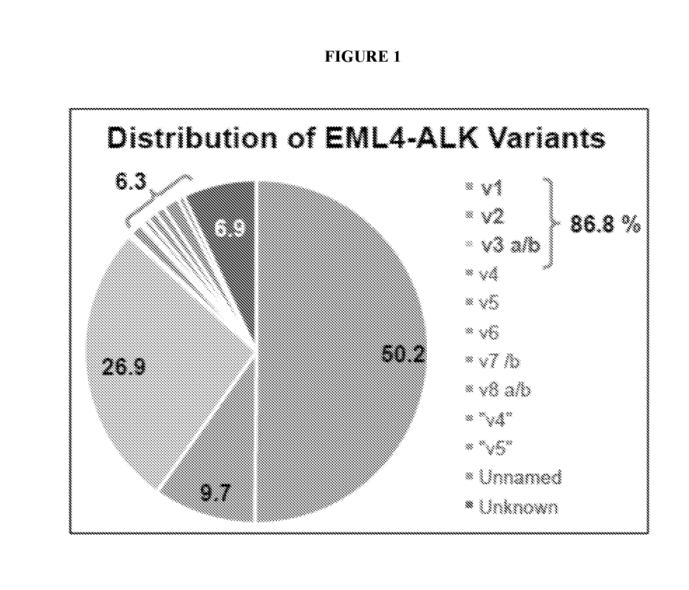

The EML4-ALK translocation is a predictive driver mutation in non-small cell lung cancer (NSCLC). EML4-ALK translocations comprise several variants, the clinical majority of which are v1, v2, and v3 (FIG. 1). As presence of these translocations determines both resistance to EGFR inhibitors and druggability with FDA-approved ALK kinase inhibitors, molecular profiling of the respective fusion transcripts is a critical prerequisite to therapy. Ongoing clinical trials and development of new ALK inhibitors for personalized treatment demand development of robust diagnostics.

Current determination of EML4-ALK fusions relies on tissue biopsies and fine-needle aspirates—techniques constrained by surgical complications, availability of tissue, and sample heterogeneity. To address the shortcomings of current tissue-based molecular profiling and to streamline the diagnostic procedure for NSCLC patients, the methods and kits described herein provide a plasma-based assay, referred to herein as “EXO501a,” to rapidly detect fusion transcripts via a single blood draw. This liquid biopsy diagnostic has the potential to provide valuable benefits for non-surgical treatment guidance and longitudinal monitoring of EML4-ALK positive patients.

Current lung cancer diagnosis is done by pathologists, and sampling tumor tissue has significant inherent limitations, such as, for example, tumor tissue is a single snapshot in time, is subject to selection bias resulting from tumor heterogeneity, and can be difficult to obtain. In some cases, a sufficient sample of tumor tissue is not available for some patients and/or obtaining a tissue sample can cause complications such as pneumothorax. However, so far, the reference non-standard method for patient stratification has been tissue biopsies.

The kits and methods provided herein leverage the ability to look at the entire disease process and the tumor environment, as there are several processes that are leading to the release of nucleic acids (extracellular RNA and DNA) into the blood stream. Amongst these processes are, for example, apoptosis and necrosis. Apoptotic or necrotic cells may release cell free DNA (cfDNA) in apoptotic vesicles or as circulating nucleosomes. Additionally, exosomes are actively released by living cells directly from the plasma membrane or via the multivesicular body pathway, carrying RNA into circulation (exoRNA). In contrast to the current methods of detecting an ALK fusion transcript, e.g., an EML4-ALK fusion transcript, in a patient sample, the methods and kits provided herein are able to analyze all of the processes that are simultaneously happening inside the tumor.

These methods and kits are novel: detecting an ALK fusion transcript, e.g., an EML4-ALK fusion transcript, in the exosomal RNA fraction is new. These methods and kits are also not obvious over current methods as it has only recently been understood, that blood contains tumor-derived RNA that can be used for diagnostic assays.

Thus, the methods and kits described herein provide a number of advantages over currently available detection methods and kits. Liquid biopsies, in contrast to tissue, represent a non-invasive and low-risk method to detect the predictive biomarker EML4-ALK in plasma of NSCLC patients at baseline and to monitor longitudinally during therapy. Furthermore, the EXO501a assay detects EML4-ALK with high specificity for individual fusion variants from the plasma of NSCLC patients on exosomal RNA. Moreover, the qPCR-based liquid biopsy assay's performance on cellular RNA exceeds that of alternative test kits. As shown in the working examples provided herein, the EXO501a assay allows for the discrete determination of the EML4-ALK v1/v2/v3 variants, respectively. Current kits on the market, however, do not allow for the discrete determination of these variants.

In some embodiments, the methods and kits provided herein is a qPCR-based EML4-ALK liquid biopsy assay that isolates and analyzes exosomal RNA (exoRNA) from plasma to provide detection of the mutation with high specificity for five distinct EML4-ALK fusion transcripts, referred to as v1, v2, v3a, b, c. These five fusion transcripts account for up to 85% of the known EML4-ALK fusions. Fusion transcript identification is increasingly important to inform targeted therapy selection.

EML4-ALK is a gene fusion found in approximately three to five percent of all patients with NSCLC. The current testing standard for EML4-ALK is FISH or IHC from a tissue biopsy. Tissue in NSCLC patients is sometimes not available. Thus, the methods and kits provided herein help serve this population who otherwise could not be tested.

These methods and kits provide a number of key benefits such as, for example, the ability to analyze stable, high-quality exoRNA to detect EML4-ALK mutation; the ability to detect with high specificity distinct fusion transcripts (v1, v2, v3a, b, c), which is increasingly important for treatment selection; the ability to conduct longitudinal testing; the ability to enable molecular analysis without the need for tissue samples and to avoid issue such as tissue scarcity and/or lack of homogeneity; and the flexibility to use either fresh or frozen/archived plasma samples from subjects.

In some embodiments, the disclosure provides a method for the diagnosis, prognosis, monitoring or therapy selection for a disease or other medical condition in a subject in need thereof by (a) providing a biological sample from a subject; (b) isolating microvesicles from the biological sample; (c) extracting one or more nucleic acids from the microvesicles; and (d) detecting the presence or absence of an ALK fusion transcript in the extracted nucleic acids, wherein the presence of the ALK fusion transcript in the extracted nucleic acids indicates the presence of a disease or other medical condition in the subject or a higher predisposition of the subject to develop a disease or other medical condition.

In some embodiments, the ALK fusion transcript is an EML4-ALK fusion transcript. In some embodiments, the EML4-ALK fusion transcript is selected from the group consisting of EML4-ALK v1, EML4-ALK v2, EML4-ALK v3a, EML4-ALK v3b, EML4-ALKv3c, and combinations thereof. In some embodiments, the EML4-ALK fusion transcript is a combination of the following EML4-ALK fusion transcripts: EML4-ALK v1, EML4-ALK v2, EML4-ALK v3a, EML4-ALK v3b, and EML4-ALKv3c.

In some embodiments, the biological sample is a bodily fluid. In some embodiments, the biological sample is plasma or serum.

In some embodiments, the disease or other medical condition is cancer. In some embodiments, the disease or other medical condition is lung cancer. In some embodiments, the disease or other medical condition is non-small cell lung cancer (NSCLC).

In some embodiments, step (c) comprises the isolation of exosomal RNA from the biological sample. In some embodiments, step (c) further comprises reverse transcription of the isolated exosomal RNA.

In some embodiments, a control nucleic acid or control particle or combination thereof is spiked into the reverse transcription reaction.

In some embodiments, step (c) further comprises a pre-amplification step following reverse transcription of the isolated exosomal RNA. In some embodiments, the pre-amplification step comprises use of a positive amplification control. In some embodiments, the positive amplification control comprises a reference DNA encoding for EML4-ALK v1, a reference DNA encoding for EML4-ALK v2, a reference DNA encoding for EML4-ALK v3, a reference DNA coding for RPL4, a reference RNA coding Qbeta, and combinations thereof. In some embodiments, the reference nucleic acid or combination of reference nucleic acids is quantified using a PCR based method. In some embodiments, the reference nucleic acid or combination of reference nucleic acids is quantified using qPCR.

In some embodiments, the pre-amplification step comprises use of a negative amplification control. In some embodiments, the negative amplification control comprises a reference DNA encoding for EML4-ALK v1, a reference DNA encoding for EML4-ALK v2, a reference DNA encoding for EML4-ALK v3, a reference DNA coding for RPL4, a reference RNA coding Qbeta, and combinations thereof. In some embodiments, the reference nucleic acid or combination of reference nucleic acids is quantified using a PCR based method wherein water is used in place of a nucleic acid template. In some embodiments, the reference nucleic acid or combination of reference nucleic acids is quantified using qPCR wherein water is used in place of a nucleic acid template.

In some embodiments, step (d) comprises a sequencing-based detection technique. In some embodiments, the sequencing-based detection technique comprises a PCR technique or a next-generation sequencing technique.

In some embodiments, step (d) further comprises detecting one or more controls. In some embodiments, the control is a housekeeping gene. In some embodiments, the housekeeping gene is RPL4. In some embodiments, the control is expression level of Qbeta spiked into the extraction of step (c).

In some embodiments, the method further comprises step (e) analyzing the data from step (d) to stratify the samples as positive or negative according to the detected level of cycle threshold (CT) values.

In some embodiments, step (d) comprises identifying the biological sample as positive when the level of EML4-ALK variant 1 is at least a cycle threshold (CT) of less than or equal to 31, the level of EML4-ALK variant 2 is at least a CT value of less than or equal to 32, and the level of EML4-ALK variant 3 is at least a CT value of less than or equal to 32.

In some embodiments, step (d) comprises identifying the biological sample as negative when at least one the following cycle threshold (CT) values is detected in the biological sample: the level of EML4-ALK variant 1 is at least a CT value of greater than or equal to 31, the level of EML4-ALK variant 2 is at least a CT value of greater than or equal to 32, and the level of EML4-ALK variant 3 is at least a CT value of greater than or equal to 32.

In some embodiments, the method further comprises step (e) analyzing the data from step (d) using machine-learning based modeling, data mining methods, and/or statistical analysis. In some embodiments, the data is analyzed to identify or predict disease outcome of the patient. In some embodiments, the data is analyzed to stratify the patient within a patient population. In some embodiments, the data is analyzed to identify or predict whether the patient is resistant to treatment with an anti-cancer therapy. In some embodiments, the data is analyzed to identify or predict whether the patient is resistant to treatment with an EGFR therapy, such as, by way of non-limiting example, treatment with an EGFR inhibitor. In some embodiments, the data is analyzed to measure progression-free survival progress of the subject. In some embodiments, the data is analyzed to select a treatment option for the subject when an EML4-ALK transcript is detected.

In some embodiments, the method further comprises administering to the subject a therapeutically effective amount of an anti-cancer therapy. In some embodiments, the treatment option is treatment with a combination of therapies.

In some embodiments, the treatment option is treatment with crizotinib (Xalkori). In some embodiments, the treatment option is treatment with ceritinib (Zykadia) or alectinib (Alecensa) if crizotinib stops working or is not well tolerated.

In some embodiments, the treatment option is treatment with an EGFR inhibitor. In some embodiments, the EGFR inhibitor is a tyrosine kinase inhibitor or a combination of tyrosine kinase inhibitors. In some embodiments, the EGFR inhibitor is a first generation tyrosine kinase inhibitor or a combination of first generation tyrosine kinase inhibitors. In some embodiments, the EGFR inhibitor is a second generation tyrosine kinase inhibitor or a combination of second generation tyrosine kinase inhibitors. In some embodiments, the EGFR inhibitor is a third generation tyrosine kinase inhibitor or a combination of third generation tyrosine kinase inhibitors. In some embodiments, the EGFR inhibitor is a combination of a first generation tyrosine kinase inhibitor, a second generation tyrosine kinase inhibitor, and/or a third generation tyrosine kinase inhibitor. In some embodiments, the EGFR inhibitor is erlotinib, gefitinib, another tyrosine kinase inhibitor, or combinations thereof.

The methods and kits described herein isolate microvesicles by capturing the microvesicles to a surface and subsequently lysing the microvesicles to release the nucleic acids, particularly RNA, contained therein. Microvesicles are shed by eukaryotic cells, or budded off of the plasma membrane, to the exterior of the cell. These membrane vesicles are heterogeneous in size with diameters ranging from about 10 nm to about 5000 nm. These microvesicles include microvesicles, microvesicle-like particles, prostasomes, dexosomes, texosomes, ectosomes, oncosomes, apoptotic bodies, retrovirus-like particles, and human endogenous retrovirus (HERV) particles. Small microvesicles (approximately 10 to 5000 nm, and more often 30 to 200 nm in diameter) that are released by exocytosis of vesicles are referred to in the art as “microvesicles.”

Microvesicles are a rich source of high quality nucleic acids, excreted by all cells and present in all human biofluids. The RNA in microvesicles provides a snapshot of the transcriptome of primary tumors, metastases and the surrounding microenvironment in real-time. Thus, accurate assessment of the RNA profile of microvesicles by assays provides companion diagnostics and real-time monitoring of disease. This development has been stalled by the current standard of isolating exosomes which is slow, tedious, variable and not suited for a diagnostic environment.

The isolation and extraction methods and/or kits provided herein use a spin-column based purification process using an affinity membrane that binds microvesicles. The isolation and extraction methods are further described in PCT Publication Nos. WO 2016/007755 and WO 2014/107571, the contents of each of which are described herein in their entirety. The methods and kits of the disclosure allow for the capability to run large numbers of clinical samples in parallel, using volumes from 0.2 up to 4 mL on a single column. The isolated RNA is highly pure, protected by a vesicle membrane until lysis, and intact vesicles can be eluted from the membrane. The isolation and extraction procedures are able to deplete all mRNA from plasma input, and are equal or better in mRNA/miRNA yield when compared to ultracentrifugation or direct lysis. In contrast, the methods and/or kits provided herein enrich for the microvesicle bound fraction of miRNAs, and they are easily scalable to large amounts of input material. This ability to scale up enables research on interesting, low abundant transcripts. In comparison with other commercially available products on the market, the methods and kits of the disclosure provide unique capabilities that are demonstrated by the examples provided herein.

The isolation of microvesicles from a biological sample prior to extraction of nucleic acids is advantageous for the following reasons: 1) extracting nucleic acids from microvesicles provides the opportunity to selectively analyze disease or tumor-specific nucleic acids obtained by isolating disease or tumor-specific microvesicles apart from other microvesicles within the fluid sample; 2) nucleic acid-containing microvesicles produce significantly higher yields of nucleic acid species with higher integrity as compared to the yield/integrity obtained by extracting nucleic acids directly from the fluid sample without first isolating microvesicles; 3) scalability, e.g., to detect nucleic acids expressed at low levels, the sensitivity can be increased by concentrating microvesicles from a larger volume of sample using the methods described herein; 4) more pure or higher quality/integrity of extracted nucleic acids in that proteins, lipids, cell debris, cells and other potential contaminants and PCR inhibitors that are naturally found within biological samples are excluded before the nucleic acid extraction step; and 5) more choices in nucleic acid extraction methods can be utilized as isolated microvesicle fractions can be of a smaller volume than that of the starting sample volume, making it possible to extract nucleic acids from these fractions or pellets using small volume column filters.

Several methods of isolating microvesicles from a biological sample have been described in the art. For example, a method of differential centrifugation is described in a paper by Raposo et al. (Raposo et al., 1996), a paper by Skog et. al. (Skog et al., 2008) and a paper by Nilsson et. al. (Nilsson et al., 2009). Methods of ion exchange and/or gel permeation chromatography are described in U.S. Pat. Nos. 6,899,863 and 6,812,023. Methods of sucrose density gradients or organelle electrophoresis are described in U.S. Pat. No. 7,198,923. A method of magnetic activated cell sorting (MACS) is described in a paper by Taylor and Gercel Taylor (Taylor and Gercel-Taylor, 2008). A method of nanomembrane ultrafiltration concentration is described in a paper by Cheruvanky et al. (Cheruvanky et al., 2007). A method of Percoll gradient isolation is described in a publication by Miranda et al. (Miranda et al., 2010). Further, microvesicles may be identified and isolated from bodily fluid of a subject by a microfluidic device (Chen et al., 2010). In research and development, as well as commercial applications of nucleic acid biomarkers, it is desirable to extract high quality nucleic acids from biological samples in a consistent, reliable, and practical manner.

Nucleic Acid Extraction

The methods disclosed herein use a highly enriched microvesicle fraction for extraction of high quality nucleic acids from said microvesicles. The nucleic acid extractions obtained by the methods described herein may be useful for various applications in which high quality nucleic acid extractions are required or preferred, such as for use in the diagnosis, prognosis, or monitoring of diseases or medical conditions, such as for example, cancer. The methods and kits provided herein are useful in detecting EML4-ALK fusion transcripts for the diagnosis of non-small cell lung cancer (NSCLC).

The quality or purity of the isolated microvesicles can directly affect the quality of the extracted microvesicle nucleic acids, which then directly affects the efficiency and sensitivity of biomarker assays for disease diagnosis, prognosis, and/or monitoring. Given the importance of accurate and sensitive diagnostic tests in the clinical field, methods for isolating highly enriched microvesicle fractions from biological samples are needed. To address this need, the present invention provides methods for isolating microvesicles from biological sample for the extraction of high quality nucleic acids from a biological sample. As shown herein, highly enriched microvesicle fractions are isolated from biological samples by methods described herein, and wherein high quality nucleic acids subsequently extracted from the highly enriched microvesicle fractions. These high quality extracted nucleic acids are useful for measuring or assessing the presence or absence of biomarkers for aiding in the diagnosis, prognosis, and/or monitoring of diseases or other medical conditions.

As used herein, the term “biological sample” refers to a sample that contains biological materials such as DNA, RNA and protein. In some embodiments, the biological sample may suitably comprise a bodily fluid from a subject. The bodily fluids can be fluids isolated from anywhere in the body of the subject, for example, a peripheral location, including but not limited to, for example, blood, plasma, serum, urine, sputum, spinal fluid, cerebrospinal fluid, pleural fluid, nipple aspirates, lymph fluid, fluid of the respiratory, intestinal, and genitourinary tracts, tear fluid, saliva, breast milk, fluid from the lymphatic system, semen, intra-organ system fluid, ascitic fluid, tumor cyst fluid, amniotic fluid and cell culture supernatant, and combinations thereof. In some embodiments, the body fluid is plasma. Suitably a sample volume of about 0.1 ml to about 30 ml fluid may be used. The volume of fluid may depend on a few factors, e.g., the type of fluid used. For example, the volume of serum samples may be about 0.1 ml to about 4 ml, for example, about 0.2 ml to 4 ml. The volume of plasma samples may be about 0.1 ml to about 4 ml, for example, 0.5 ml to 4 ml. The volume of urine samples may be about 10 ml to about 30 ml, for example, about 20 ml. Biological samples can also include fecal or cecal samples, or supernatants isolated therefrom.

The term “subject” is intended to include all animals shown to or expected to have nucleic acid-containing particles. In particular embodiments, the subject is a mammal, a human or nonhuman primate, a dog, a cat, a horse, a cow, other farm animals, or a rodent (e.g. mice, rats, guinea pig. etc.). A human subject may be a normal human being without observable abnormalities, e.g., a disease. A human subject may be a human being with observable abnormalities, e.g., a disease. The observable abnormalities may be observed by the human being himself, or by a medical professional. The term “subject,” “patient,” and “individual” are used interchangeably herein.

As used herein, the term “nucleic acids” refer to DNA and RNA. The nucleic acids can be single stranded or double stranded. In some instances, the nucleic acid is DNA. In some instances, the nucleic acid is RNA. RNA includes, but is not limited to, messenger RNA, transfer RNA, ribosomal RNA, non-coding RNAs, microRNAs, and HERV elements.

In some embodiments, a high quality nucleic acid extraction is an extraction in which one is able to detect 18S and 28S rRNA. In some embodiments, the quantification of 18S and 28S rRNAs extracted can be used determine the quality of the nucleic acid extraction. In some embodiments, the quantification of 18S and 28S rRNA is in a ratio of approximately 1:1 to approximately 1:2; for example, approximately 1:2. Ideally, high quality nucleic acid extractions obtained by the methods described herein will also have an RNA integrity number of greater than or equal to 5 for a low protein biological sample (e.g., urine), or greater than or equal to 3 for a high protein biological sample (e.g., serum), and a nucleic acid yield of greater than or equal to 50 pg/ml from a 20 ml low protein biological sample or a 1 ml high protein biological sample.

High quality RNA extractions are desirable because RNA degradation can adversely affect downstream assessment of the extracted RNA, such as in gene expression and mRNA analysis, as well as in analysis of non-coding RNA such as small RNA and microRNA. The new methods described herein enable one to extract high quality nucleic acids from microvesicles isolated from a biological sample so that an accurate analysis of nucleic acids within the microvesicles can be performed.

Following the isolation of microvesicles from a biological sample, nucleic acid may be extracted from the isolated or enriched microvesicle fraction. To achieve this, in some embodiments, the microvesicles may first be lysed. The lysis of microvesicles and extraction of nucleic acids may be achieved with various methods known in the art, including those described in PCT Publication Nos. WO 2016/007755 and WO 2014/107571, the contents of each of which are hereby incorporated by reference in their entirety. Such methods may also utilize a nucleic acid-binding column to capture the nucleic acids contained within the microvesicles. Once bound, the nucleic acids can then be eluted using a buffer or solution suitable to disrupt the interaction between the nucleic acids and the binding column, thereby successfully eluting the nucleic acids.

In some embodiments, the nucleic acid extraction methods also include the step of removing or mitigating adverse factors that prevent high quality nucleic acid extraction from a biological sample. Such adverse factors are heterogeneous in that different biological samples may contain various species of adverse factors. In some biological samples, factors such as excessive DNA may affect the quality of nucleic acid extractions from such samples. In other samples, factors such as excessive endogenous RNase may affect the quality of nucleic acid extractions from such samples. Many agents and methods may be used to remove these adverse factors. These methods and agents are referred to collectively herein as an “extraction enhancement operations.” In some instances, the extraction enhancement operation may involve the addition of nucleic acid extraction enhancement agents to the biological sample. To remove adverse factors such as endogenous RNases, such extraction enhancement agents as defined herein may include, but are not limited to, an RNase inhibitor such as Superase-In (commercially available from Ambion Inc.) or RNaselNplus (commercially available from Promega Corp.), or other agents that function in a similar fashion; a protease (which may function as an RNase inhibitor); DNase; a reducing agent; a decoy substrate such as a synthetic RNA and/or carrier RNA; a soluble receptor that can bind RNase; a small interfering RNA (siRNA); an RNA binding molecule, such as an anti-RNA antibody, a basic protein or a chaperone protein; an RNase denaturing substance, such as a high osmolarity solution, a detergent, or a combination thereof.

For example, the extraction enhancement operation may include the addition of an RNase inhibitor to the biological sample, and/or to the isolated microvesicle fraction, prior to extracting nucleic acid; for example, in some embodiments, the RNase inhibitor has a concentration of greater than 0.027 AU (1×) for a sample equal to or more than 1 μl in volume; alternatively, greater than or equal to 0.135 AU (5×) for a sample equal to or more than 1 μl; alternatively, greater than or equal to 0.27 AU (10×) for a sample equal to or more than I μl; alternatively, greater than or equal to 0.675 AU (25×) for a sample equal to or more than 1 μl; and alternatively, greater than or equal to 1.35 AU (50×) for a sample equal to or more than 1 μl; wherein the 1× concentration refers to an enzymatic condition wherein 0.027 AU or more RNase inhibitor is used to treat microvesicles isolated from 1 μl or more bodily fluid, the 5× concentration refers to an enzymatic condition wherein 0.135 AU or more RNase inhibitor is used to treat microvesicles isolated from 1 μl or more bodily fluid, the 10× protease concentration refers lo an enzymatic condition wherein 0.27 AU or more RNase inhibitor is used to treat particles isolated from 1 μl or more bodily fluid, the 25× concentration refers to an enzymatic condition wherein 0.675 AU or more RNase inhibitor is used to treat microvesicles isolated from 1 μl or more bodily fluid, and the 50× protease concentration refers to an enzymatic condition wherein 1.35 AU or more RNase inhibitor is used to treat particles isolated from 1 μl or more bodily fluid. In some embodiments, the RNase inhibitor is a protease, in which case, 1 AU is the protease activity that releases folin-positive amino acids and peptides corresponding to 1 μmol tyrosine per minute.

These enhancement agents may exert their functions in various ways, e.g., through inhibiting RNase activity (e.g., RNase inhibitors), through a ubiquitous degradation of proteins (e.g., proteases), or through a chaperone protein (e.g., a RNA-binding protein) that binds and protects RNAs. In all instances, such extraction enhancement agents remove or at least mitigate some or all of the adverse factors in the biological sample or associated with the isolated particles that would otherwise prevent or interfere with the high quality extraction of nucleic acids from the isolated particles.

Detection of Nucleic Acid Biomarkers

The analysis of nucleic acids present in the isolated particles is quantitative and/or qualitative. For quantitative analysis, the amounts (expression levels), either relative or absolute, of specific nucleic acids of interest within the isolated particles are measured with methods known in the art (described below). For qualitative analysis, the species of specific nucleic acids of interest within the isolated microvesicles, whether wild type or variants, are identified with methods known in the art.

The present invention also includes various uses of the new methods of isolating microvesicles from a biological sample for high quality nucleic acid extraction from a for (i) aiding in the diagnosis of a subject, (ii) monitoring the progress or reoccurrence of a disease or other medical condition in a subject, or (iii) aiding in the evaluation of treatment efficacy for a subject undergoing or contemplating treatment for a disease or other medical condition; wherein the presence or absence of one or more biomarkers in the nucleic acid extraction obtained from the method is determined, and the one or more biomarkers are associated with the diagnosis, progress or reoccurrence, or treatment efficacy, respectively, of a disease or other medical condition.

In some embodiments, it may be beneficial or otherwise desirable to amplify the nucleic acid of the microvesicle prior to analyzing it. Methods of nucleic acid amplification are commonly used and generally known in the art, many examples of which are described herein. If desired, the amplification can be performed such that it is quantitative. Quantitative amplification will allow quantitative determination of relative amounts of the various nucleic acids, to generate a genetic or expression profile.

In some embodiments, the extracted nucleic acid comprises RNA. In this instance, the RNA is reverse-transcribed into complementary DNA (cDNA) before further amplification. Such reverse transcription may be performed alone or in combination with an amplification step. One example of a method combining reverse transcription and amplification steps is reverse transcription polymerase chain reaction (RT-PCR), which may be further modified to be quantitative, e.g., quantitative RT-PCR as described in U.S. Pat. No. 5,639,606, which is incorporated herein by reference for this teaching. Another example of the method comprises two separate steps: a first of reverse transcription to convert RNA into cDNA and a second step of quantifying the amount of cDNA using quantitative PCR. As demonstrated in the examples that follow, the RNAs extracted from nucleic acid-containing particles using the methods disclosed herein include many species of transcripts including, but not limited to, ribosomal 18S and 28S rRNA, microRNAs, transfer RNAs, transcripts that are associated with diseases or medical conditions, and biomarkers that are important for diagnosis, prognosis and monitoring of medical conditions.

For example, RT-PCR analysis determines a CT (cycle threshold) value for each reaction. In RT-PCR, a positive reaction is detected by accumulation of a fluorescence signal. The CT value is defined as the number of cycles required for the fluorescent signal to cross the threshold (i.e., exceeds background level). CT levels are inversely proportional to the amount of target nucleic acid, or control nucleic acid, in the sample (i.e., the lower the CT level, the greater the amount of control nucleic acid in the sample).

In another embodiment, the copy number of the control nucleic acid can be measured using any of a variety of art-recognized techniques, including, but not limited to, RT-PCR. Copy number of the control nucleic acid can be determined using methods known in the art, such as by generating and utilizing a calibration, or standard curve.

In some embodiments, one or more biomarkers can be one or a collection of genetic aberrations, which is used herein to refer to the nucleic acid amounts as well as nucleic acid variants within the nucleic acid-containing particles. Specifically, genetic aberrations include, without limitation, transcript variants, over-expression of a gene (e.g., an oncogene) or a panel of genes, under-expression of a gene (e.g., a tumor suppressor gene such as p53 or RB) or a panel of genes, alternative production of splice variants of a gene or a panel of genes, gene copy number variants (CNV) (e.g., DNA double minutes) (Hahn, 1993), nucleic acid modifications (e.g., methylation, acetylation and phosphorylations), single nucleotide polymorphisms (SNPs), chromosomal rearrangements (e.g., inversions, deletions and duplications), and mutations (insertions, deletions, duplications, missense, nonsense, synonymous or any other nucleotide changes) of a gene or a panel of genes, which mutations, in many cases, ultimately affect the activity and function of the gene products, lead to alternative transcriptional splice variants and/or changes of gene expression level, or combinations of any of the foregoing.

Nucleic acid amplification methods include, without limitation, polymerase chain reaction (PCR) (U.S. Pat. No. 5,219,727) and its variants such as in situ polymerase chain reaction (U.S. Pat. No. 5,538,871), quantitative polymerase chain reaction (U.S. Pat. No. 5,219,727), nested polymerase chain reaction (U.S. Pat. No. 5,556,773), self-sustained sequence replication and its variants (Guatelli et al., 1990), transcriptional amplification system and its variants (Kwoh et al., 1989), Qb Replicase and its variants (Miele et al., 1983), cold-PCR (Li et al., 2008), BEAMing (Li et al., 2006) or any other nucleic acid amplification methods, followed by the detection of the amplified molecules using techniques well known to those of skill in the art. Especially useful are those detection schemes designed for the detection of nucleic acid molecules if such molecules are present in very low numbers. The foregoing references are incorporated herein for their teachings of these methods. In other embodiment, the step of nucleic acid amplification is not performed. Instead, the extract nucleic acids are analyzed directly (e.g., through next-generation sequencing).

The determination of such genetic aberrations can be performed by a variety of techniques known to the skilled practitioner. For example, expression levels of nucleic acids, alternative splicing variants, chromosome rearrangement and gene copy numbers can be determined by microarray analysis (see, e.g., U.S. Pat. Nos. 6,913,879, 7,364,848, 7,378,245, 6,893,837 and 6,004,755) and quantitative PCR. Particularly, copy number changes may be detected with the Illumina Infinium II whole genome genotyping assay or Agilent Human Genome CGH Microarray (Steemers et al., 2006). Nucleic acid modifications can be assayed by methods described in, e.g., U.S. Pat. No. 7,186,512 and patent publication WO2003/023065. Particularly, methylation profiles may be determined by Illumina DNA Methylation OMA003 Cancer Panel. SNPs and mutations can be detected by hybridization with allele-specific probes, enzymatic mutation detection, chemical cleavage of mismatched heteroduplex (Cotton et al., 1988), ribonuclease cleavage of mismatched bases (Myers et al., 1985), mass spectrometry (U.S. Pat. Nos. 6,994,960, 7,074,563, and 7,198,893), nucleic acid sequencing, single strand conformation polymorphism (SSCP) (Orita et al., 1989), denaturing gradient gel electrophoresis (DGGE)(Fischer and Lerman, 1979a; Fischer and Lerman, 1979b), temperature gradient gel electrophoresis (TGGE) (Fischer and Lerman, 1979a; Fischer and Lerman, 1979b), restriction fragment length polymorphisms (RFLP) (Kan and Dozy, 1978a; Kan and Dozy, 1978b), oligonucleotide ligation assay (OLA), allele-specific PCR (ASPCR) (U.S. Pat. No. 5,639,611), ligation chain reaction (LCR) and its variants (Abravaya et al., 1995; Landegren et al., 1988; Nakazawa et al., 1994), flow-cytometric heteroduplex analysis (WO/2006/113590) and combinations/modifications thereof. Notably, gene expression levels may be determined by the serial analysis of gene expression (SAGE) technique (Velculescu et al., 1995). In general, the methods for analyzing genetic aberrations are reported in numerous publications, not limited to those cited herein, and are available to skilled practitioners. The appropriate method of analysis will depend upon the specific goals of the analysis, the condition/history of the patient, and the specific cancer(s), diseases or other medical conditions to be detected, monitored or treated. The forgoing references are incorporated herein for their teaching of these methods.

Many biomarkers may be associated with the presence or absence of a disease or other medical condition in a subject. Therefore, detection of the presence or absence of ELK4-AKL fusion transcripts in a nucleic acid extraction from isolated particles, according to the methods disclosed herein, aid diagnosis of a disease or other medical condition such as NSCLC in the subject.

Further, many biomarkers may help disease or medical status monitoring in a subject. Therefore, the detection of the presence or absence of such biomarkers in a nucleic acid extraction from isolated particles, according to the methods disclosed herein, may aid in monitoring the progress or reoccurrence of a disease or other medical condition in a subject.

Many biomarkers have also been found to influence the effectiveness of treatment in a particular patient. Therefore, the detection of the presence or absence of such biomarkers in a nucleic acid extraction from isolated particles, according to the methods disclosed herein, may aid in evaluating the efficacy of a given treatment in a given patient. The identification of these biomarkers in nucleic acids extracted from isolated particles from a biological sample from a patient may guide the selection of treatment for the patient.

In certain embodiments of the foregoing aspects of the invention, the disease or other medical condition is a neoplastic disease or condition (e.g., cancer or cell proliferative disorder). In some embodiments, the disease or other medical condition is a lung cancer. In some embodiments, the disease or other medical condition is non-small cell lung cancer (NSCLC).

Kits for Isolating Microvesicles from a Biological Sample

One aspect of the present invention is further directed to kits for use in the methods disclosed herein. The kit comprises a capture surface apparatus sufficient to separate microvesicles from a biological sample from unwanted particles, debris, and small molecules that are also present in the biological sample, and a means for detecting ELK4-ALK fusion transcripts. The present invention also optionally includes instructions for using the foregoing reagents in the isolation and optional subsequent nucleic acid extraction process.

### EXAMPLES

### Example 1: EXO501a Assay Workflow

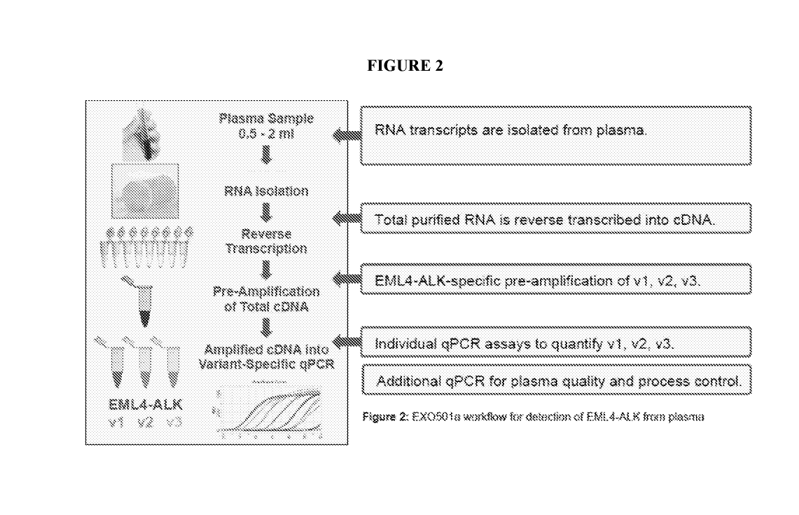

FIG. 2 is a flowchart that depicts the workflow of the EXO501a assay for detection of EML4-ALK fusion transcripts from plasma of lung cancer patients (NSCLC). The EXO501a assay is advantageous because it allows for variant-specific detection of various EML4-ALK fusion transcripts such as v1/v2/v3 a,b,c. Furthermore, the assay is both specific, as no false positive detection of ALK wt or fusion (based on ref RNA) has been detected using this assay, and sensitive, as five copies of ref RNA have been found in a 2 ml plasma sample.

Using EXO501a consistently and reproducibly isolated sufficient amounts of high-quality microvesicle RNA (i.e., RNA extracted from the microvesicle fraction of a plasma sample) from a few milliliters of NSCLC patient plasma for analysis and quantification of EML4-ALK fusions.

Additionally, in some embodiments, the EXO501a can be run using controls. For example, in some embodiments, the plasma samples are analyzed for reference genes that are used as indicators of the plasma quality. In some embodiments, the reference gene(s) is/are a plasma-inherent transcript. In some embodiments, the reference gene(s) is/are selected from the group consisting of EML4, RPL4, NDUFA1, and any combinations thereof. In some embodiments, the reference gene(s) is/are analyzed by additional qPCR.

Additional controls that can be used in the EXO501a assay include in-process controls for reverse transcriptase and/or PCR performance. These in-process controls include, by way of non-limiting examples, a reference RNA (also referred to herein as ref.RNA), that is spiked in after RNA isolation and prior to reverse transcription. In some embodiments, the ref RNA is a control such as Qbeta. In some embodiments, the ref RNA is analyzed by additional PCR.

### Example 2: EXO501a Analysis of Patient Samples

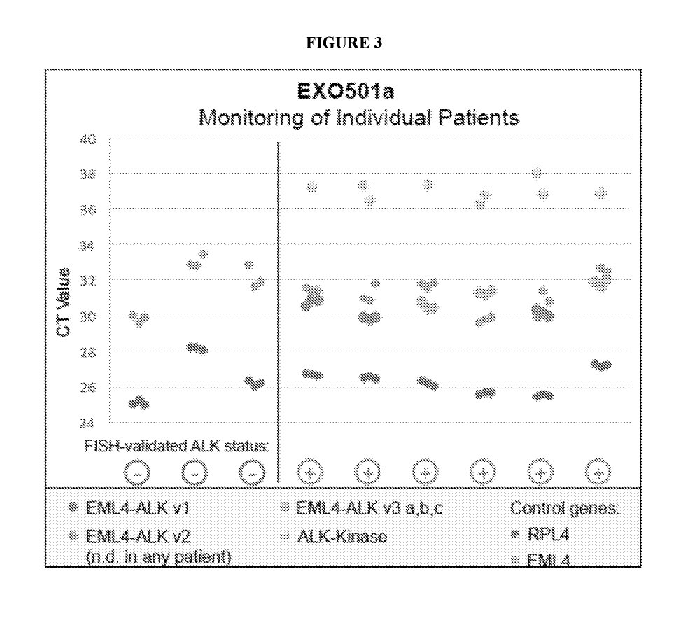

The EXO501a assay was validated on non-small cell lung cancer (NSCLC) patients. Exemplary results are shown in FIG. 3. As a proof of concept, tissue-correlated plasma samples were analyzed for the presence of the EML4-ALK v1/v2/v3 variants, respectively.

Additionally, positive plasma samples were confirmed by qPCR for increased ALK expression. In a cohort of 29 patients, no false positive samples were detected; true positive concordance will be determined on an increased number of defined patient samples.

### Example 3: Evaluation of EXO501a Assay Performance

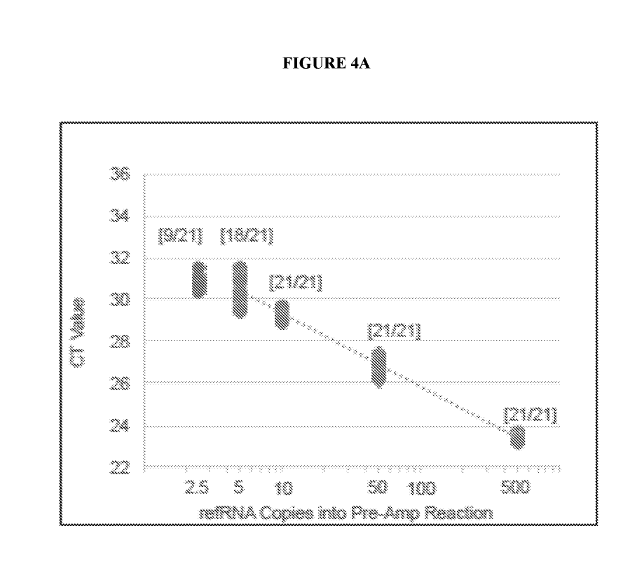

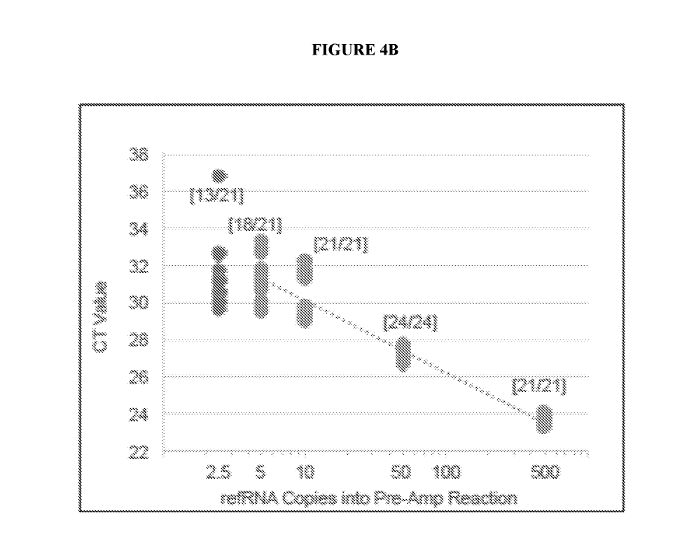

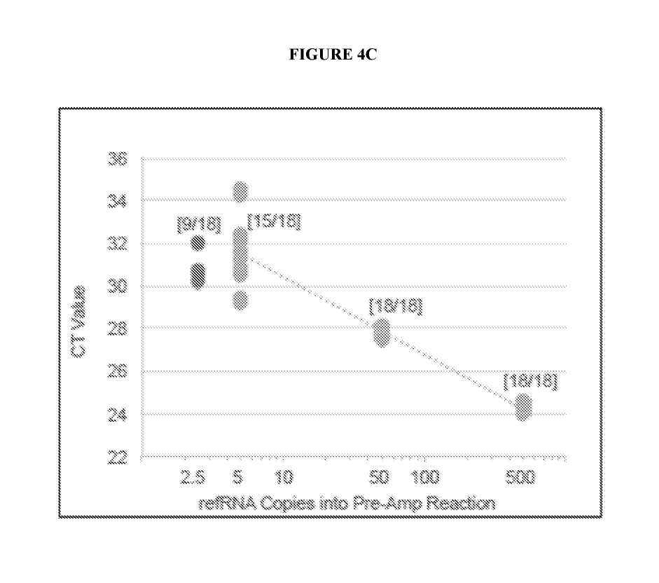

The reproducibility and sensitivity of the EXO501a assay was evaluated for each variant of EML4-ALK fusion transcript by applying synthetic reference RNA spiked into healthy patient plasma at the RT step of the workflow shown in FIG. 2. The results of this analysis are shown in FIG. 4.

Limit of detection (LOD) was determined as 2.5 copies per reaction. Assay specificity was identified as 100% for variant-specific detection of EML4-ALK, efficiency of qPCR is ranging between 92-100%.

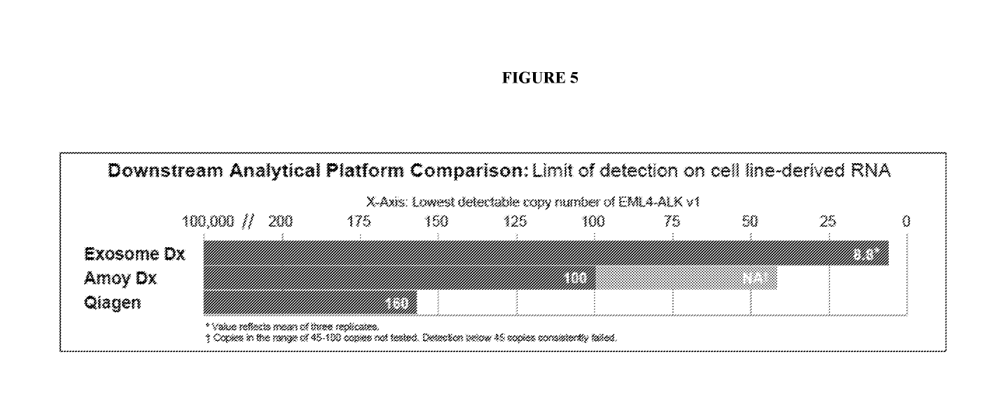

Additionally, the performance of the EXO501a assay as a downstream analytical platform was evaluated and compared to two commercially available tests. Using total RNA of an EML4-ALK v1 expressing cell line, EXO501a was compared with two commercially available tests for EML4/ALK detection: Amoy Diagnostics and Qiagen (FIG. 5). Monitoring the limit of detection, superior performance of EXO501a over the competitors for EML4-ALK v1-specific analysis was observed.

The performance of the EXO501a assay can be evaluated in many other ways, including comparison of the EXO501a assay with techniques such as FISH (fluorescence in situ hybridization).

### Example 4: EXO501a qPCR for Detection of EML4-ALK Fusion Variants

The EXO501a assay was developed for variant-specific detection of EML4-ALK fusions v1, v2, v3(a,b,c), respectively.

EML4-ALK fusions can be detected by qPCR methods using any oligonucleotide primer pair with one oligonucleotide binding to the variant-determining sequence of EML4 and the second oligonucleotide binding specifically to the sequence of ALK exon 21-exon29. The target regions for the EML4-ALK fusion variants are shown below in Table 1.

TABLE 1
Primer Design Targets

| Fusion Variant | Exons Covering Fusion Breakpoint |
|---|---|
| EML4-ALK v1 | EML4 exon13/ALK exon20 |
| EML4-ALK v2 | EML4 exon20/ALK exon20 |
| EML4-ALK v3a | EML4 exon5-exon6/ALK exon20 |
| EML4-ALK v3b | EML4 exon5-exon6-intron6(33nt insertion)/ALK |
| | exon20 |
| EML4-ALK v3c | EML4 exon5-exon6/ALK intron19(18nt insertion)- |
| | exon20 |

Selected targets and designs oligonucleotide primer and probes for qPCR detection of each variant are shown in Table 2.

In some embodiments, qPCR detection of EML4-ALK v1 is performed using the combination of primers #1, #8 and probe #24 as defined in Table 2.

In some embodiments, qPCR detection of EML4-ALK v2 is performed using the combination of primers #1, #9 and probe #24 as defined in Table 2.

In some embodiments, qPCR detection of EML4-ALK v3 is performed using the combination of primers #1, #10 and probe #24 as defined in Table 2.

TABLE 2
Target Regions of Primers

| Oligonu- | | | |
|---|---|---|---|
| cleotide | Nucleotide | Binding site | Detection of specific |
| Number # | Characteristic | on EML4-ALK | EML4-ALK Fusion Variant |
| 1 | reverse primer | ALK exon20 | defined by forward primer |
| 2 | reverse primer | ALK exon20 | defined by forward primer |
| 3 | reverse primer | ALK exon20 | defined by forward primer |
| 4 | reverse primer | ALK exon20 | defined by forward primer |
| 5 | reverse primer | ALK exon20 | defined by forward primer |
| 8 | forward primer | EML4 exon13 | EML4-ALK v1 |
| 9 | forward primer | EML4 exon20 | EML4-ALK v2 |
| 10 | forward primer | EML4 exon5 | EML4-AK v3 |
| 11 | forward primer | EML4 exon13 | EML4-ALK v1 |
| 12 | forward primer | EML4 exon5/6 | EML4-AK v3 |
| 13 | forward primer | EML4exon6 | EML4-ALK v2 |
| | | ALKexon20 | |
| 14 | forward primer | EML4exon13 | EML4-ALK v2 |
| | | ALKexon20 | |
| 15 | forward primer | EML4exon6 | EML4-ALK v2 |
| | | ALKexon20 | |
| 16 | forward primer | EML4exon13 | EML4-ALK v2 |
| | | ALKexon20 | |
| 17 | forward primer | EML4 exon13 | EML4-ALK v1 |
| 18 | forward primer | EML4 exon20 | EML4-ALK v2 |
| | | ALKexon20 | |
| 19 | forward primer | EML4 exon20 | EML4-ALK v2 |
| 20 | forward primer | EML4 exon5/6 | EML4-AK v3 |
| 21 | forward primer | EML4 exon5/6 | EML4-AK v3 |
| 22 | forward primer | EML4 exon6 | EML4-AK v3 |
| 23 | probe | ALK exon20 | defined by primer |
| 24 | probe | ALK exon20 | defined by primer |
| 25 | probe | ALK exon20 | defined by primer |
| 26 | probe | ALK exon20 | defined by primer |

### Example 5: EXO501a Algorithm for Definition of the Test Result

The EXO501a assay uses a defined algorithm to determine the result for presence/absence of EML4-ALK fusion variants 1, 2, 3(a,b,c), respectively:

Step 1:

Each sample is checked for passing the acceptance criteria for the Sample Integrity Control and the Sample Inhibition Control.

In some embodiments, the Sample Integrity Control is the expression level of the housekeeping gene RPL4 tested by qPCR.

For RPL4 the acceptance criteria are defined by a CT value ≤28.

In some embodiments, the Sample Inhibition Control is the expression level of Qbeta RNA spiked into the reverse transcription reaction of each sample and tested by qPCR.

For Qbeta RNA the acceptance criteria are defined by a CT value ≤34 for 12,500 copies spiked into reverse transcription reaction.

Step 2:

Each run of samples is checked for a set of Positive Amplification Controls being tested in parallel.

In some embodiments, the Positive Amplification Controls are defined by 3 reference DNAs coding for EML4-ALK v1, v2 v3, 1 reference DNA coding for RPL4, 1 reference RNA coding Qbeta. These reference nucleic acids are quantified by qPCR methods.

For EML4-ALK DNA the acceptance criteria are defined by a CT range of 22-25 for 50 copies of each DNA spiked into reverse transcription reaction.

For RPL4 DNA the acceptance criteria are defined by a CT range of 26-28 for 125,000 copies of DNA spiked into reverse transcription reaction.

For Qbeta RNA the acceptance criteria are defined by a CT range of 28-31 for 12,500 copies of RNA spiked into reverse transcription reaction.

Step 3:

Each run of samples is checked for a set of Negative Amplification Controls being tested in parallel.

In some embodiments, the Negative Amplification Controls are defined by the same set of qPCR as for Positive Amplification Control, but water is used instead of the nucleic acid template.

As acceptance criteria, no CT value must be detected.

If all sample-internal and external controls are passed, the sample is checked for EML4-ALK 4→Step 4.

If a sample-internal or external controls fails, the sample must be reported as “Inconclusive”. If residual sample material is available, the test is repeated from Step 1.

Step 4:

Each sample is checked for passing the acceptance criteria for expression of EML4-ALK fusion variants.

For qPCR of EML4-ALK variant 1 the acceptance criteria are CT≤31

For qPCR of EML4-ALK variant 2 the acceptance criteria are CT≤32

For qPCR of EML4-ALK variant 3 the acceptance criteria are CT≤32

If a sample passes the acceptance criteria it is reported as “Positive” for this EML4-ALK variant. The presence of variants is expected to be mutually exclusive.

If a sample fails the acceptance criteria for EML4-ALK it is reported as “Negative”.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following.

1. A method for the diagnosis, prognosis, monitoring or therapy selection for a disease or other medical condition in a subject in need thereof, the method comprising the steps of:
(a) isolating microvesicles from a biological sample from the subject;
(b) extracting one or more nucleic acids from the microvesicles; and
(c) detecting the presence or absence of an EML4-ALK fusion transcript in the extracted nucleic acids,
wherein the presence of the EML4-ALK fusion transcript in the extracted nucleic acids indicates the presence of a disease or other medical condition in the subject or a higher predisposition of the subject to develop a disease or other medical condition.

2. (canceled)

3. The method of claim 1, wherein the EML4-ALK fusion transcript is selected from the group consisting of EML4-ALK v1, EML4-ALK v2, EML4-ALK v3a, EML4-ALK v3b, EML4-ALKv3c, and combinations thereof.

4. The method of claim 1, wherein the biological sample is a bodily fluid.

5. The method of claim 1, wherein the biological sample is plasma or serum.

6. The method of claim 1, wherein the disease or other medical condition is cancer.

7. The method of claim 1, wherein the disease or other medical condition is lung cancer.

8. The method of claim 1, wherein the disease or other medical condition is non-small cell lung cancer (NSCLC).

9. The method of claim 1, wherein step (b) comprises the isolation of exosomal RNA from the biological sample.

10. The method of claim 9, wherein step (b) further comprises reverse transcription of the isolated exosomal RNA.

11. The method of claim 10, wherein a control nucleic acid or control particle or combination thereof is spiked into the reverse transcription reaction.

12. The method of claim 10, wherein step (b) comprises a pre-amplification step following reverse transcription of the isolated exosomal RNA.

13. The method of claim 12, wherein the pre-amplification step comprises use of a positive amplification control.

14. The method of claim 13, wherein the positive amplification control comprises a reference DNA encoding for EML4-ALK v1, a reference DNA encoding for EML4-ALK v2, a reference DNA encoding for EML4-ALK v3, a reference DNA coding for RPL4, a reference RNA coding Qbeta, and combinations thereof.

15. The method of claim 14, wherein the reference nucleic acid or combination of reference nucleic acids is quantified using a PCR based method.

16. The method of claim 15, wherein the reference nucleic acid or combination of reference nucleic acids is quantified using qPCR.

17. The method of claim 12, wherein the pre-amplification step comprises use of a negative amplification control.

18. The method of claim 17, wherein the negative amplification control comprises a reference DNA encoding for EML4-ALK v1, a reference DNA encoding for EML4-ALK v2, a reference DNA encoding for EML4-ALK v3, a reference DNA coding for RPL4, a reference RNA coding Qbeta, and combinations thereof.

19. The method of claim 18, wherein the reference nucleic acid or combination of reference nucleic acids is quantified using a PCR based method wherein water is used in place of a nucleic acid template.

20. The method of claim 19, wherein the reference nucleic acid or combination of reference nucleic acids is quantified using qPCR wherein water is used in place of a nucleic acid template.

21. The method of claim 1, wherein step (c) comprises a sequencing-based detection technique.

22. The method of claim 21, wherein the sequencing-based detection technique comprises a PCR technique or a next-generation sequencing technique.

23. The method of claim 1, wherein step (c) further comprises detecting one or more controls.

24. The method of claim 23, wherein the control is a housekeeping gene.

25. The method of claim 24, wherein the housekeeping gene is RPL4.

26. The method of claim 23, wherein the control is expression level of Qbeta spiked into the extraction of step (b).

27. The method of claim 1, wherein the method further comprises step (d) analyzing the data from step (c) to stratify the samples as positive or negative according to the detected level of cycle threshold (CT) values.

28. The method of claim 27, wherein step (c) comprises identifying the biological sample as positive when the level of EML4-ALK variant 1 is at least a cycle threshold (CT) of less than or equal to 31, the level of EML4-ALK variant 2 is at least a CT value of less than or equal to 32, and the level of EML4-ALK variant 3 is at least a CT value of less than or equal to 32.

29. The method of claim 27, wherein step (c) comprises identifying the biological sample as negative when at least one the following cycle threshold (CT) values is detected in the biological sample: the level of EML4-ALK variant 1 is at least a CT value of greater than or equal to 31, the level of EML4-ALK variant 2 is at least a CT value of greater than or equal to 32, and the level of EML4-ALK variant 3 is at least a CT value of greater than or equal to 32.

30. The method of claim 1, wherein the method further comprises step (d) analyzing the data from step (c) using machine-learning based modeling, data mining methods, and/or statistical analysis.

31. The method of claim 1, wherein the data is analyzed to identify or predict disease outcome of the patient.

32. The method of claim 1, wherein the data is analyzed to stratify the patient within a patient population.

33. The method of claim 1, wherein the data is analyzed to identify or predict whether the patient is resistant to treatment with an anti-cancer therapy.

34. The method of claim 1, wherein the data is analyzed to measure progression-free survival progress of the subject.

35. The method of claim 1, wherein the data is analyzed to select a treatment option for the subject when an EML4-ALK transcript is detected.

36. The method of claim 1, wherein the method further comprises administering to the subject a therapeutically effective amount of an anti-cancer therapy.
